(19) United States
(12) Patent Application Publication
(10) Pub. No.: US 2022/0099675 A1
(43) Pub. Date: Mar. 31, 2022

(54) METHODS, DEVICES, AND RELATED ASPECTS FOR DETECTING EBOLA VIRUS

(71) Applicant: ARIZONA BOARD OF REGENTS ON BEHALF OF ARIZONA STATE UNIVERSITY, Scottsdale, AZ (US)

(72) Inventors: Xiahui CHEN, Mesa, AZ (US); Shoukai KANG, Seattle, WA (US); Md Ashif IKBAL, Tempe, AZ (US); Zhi ZHAO, Mesa, AZ (US); Liangcai GU, Seattle, WA (US); Chao WANG, Chandler, AZ (US)

(21) Appl. No.: 17/489,273

(22) Filed: Sep. 29, 2021

Related U.S. Application Data
(60) Provisional application No. 63/085,004, filed on Sep. 29, 2020.

(51) Int. Cl. G01N 33/569; G01N 33/543; G01N 21/78

(52) U.S. Cl. CPC G01N 33/56983; G01N 33/54326; G01N 2469/10; G01N 21/78; G01N 2333/08; G01N 33/54346

(57) ABSTRACT

Provided herein are methods of detecting Ebola virus in a sample. The methods include contacting the sample with a plurality of gold nanoparticles (AuNPs) that are conjugated with at least two sets of antibodies, or antigen binding portions thereof, that binds to at least first or second epitopes of glycoproteins, such as secreted glycoproteins (sGPs) from the Ebola virus under conditions sufficient for the antibodies, or the antigen binding portions thereof, to bind to the first or second epitopes of the glycoproteins from the Ebola virus in the sample to produce bound glycoproteins. The methods also include detecting the glycoproteins from the Ebola virus when aggregations of the bound glycoproteins form with one another. Related reaction mixtures, devices, kits, and systems are also provided.

### CROSS-REFERENCE TO RELATED APPLICATIONS

This application claims priority to U.S. Provisional Patent Application Ser. No. 63/085,004, filed Sep. 29, 2020, the disclosure of which is incorporated herein by reference.

### STATEMENT REGARDING FEDERALLY SPONSORED RESEARCH OR DEVELOPMENT

This invention was made with government support under 1809997, 1847324, 2020464, and 1542160 awarded by the National Science Foundation and R35 GM128918 awarded by the National Institutes of Health. The government has certain rights in the invention.

### BACKGROUND

Zaire Ebolavirus (EBOV), a negative RNA-strand virus classified into Filovirade family with four other known etiological subspecies, is the pathogen that causes hemorrhagic Ebola virus disease (EVD) with statistical fatality rate ranging from 45% to 90%. Zaire Ebolavirus accounted for the first EVD epidemic in 1976 and the most massive 2014-2016 outbreak in West Africa, which caused 28,646 infected cases and 11,323 deaths. In a recent massive epidemic as of 6 Aug. 2019, another 2,781 cases and 1,866 deaths have been reported in Democratic Republic of the Congo. On 1 Jun. 2020, the World Health Organization (WHO) declared the 11^{th} outbreak ongoing in Democratic Republic of the Congo. Accumulated knowledge of EBOV outbreaks in recent decades indicates that the outbreak mode is sporadic and fast spreading, accompanied with fast virus genome evolution and high local fatality rate in underdeveloped regions. Although potential antivirus medicine, therapeutic antibodies and vaccines have been reported, there is no approved antiviral drugs by US Food and Drug Administration (FDA) and supportive care remains the only option for most treatments. These combined facts highlight the stringent diagnostic requirements in fast screening and accurate detection of EBOV for current and future epidemic preparedness and rapid, effective containment of EBOV outbreak.

Currently, most commercially available and FDA or WHO approved EVD diagnostics are based on reverse-transcription polymerase chain reaction (RT-PCR) assay, enzyme-linked immunosorbent assay (ELISA) and colloidal gold or fluorescence based rapid immunoassay. RT-PCR and ELISA generally require skilled medical practitioners operating bulky equipment and following elaborate diagnostics protocols that can takes up to 5 hours to complete. In addition, centralized laboratories with well-established EVD diagnostics capacities are generally greatly limited due to the strict biosafety regulations (BSL-4), which significantly reduce the diagnostics throughput and increase diagnostic cost taking into account the biological sample shipment charges from the epidemic region to the laboratories. These methods, although widely used in clinical diagnostics, generally lack the stringent requirements of quick, user-friendly, electricity-free and low cost in field point-of-care (POC) solutions to EVD diagnostics in medical resource limited regions.

Rapid lateral flow immunochromatographic technology has been widely adopted in POC applications. It generally meets POC requirements such as fast screening speed, low cost and zero electricity consumption. However, its detection sensitivity is relatively low (up to hundreds of ng/ml in antigen protein detection) compared to RT-PCR and ELISA and entails qualitative analysis unless coupled to readout devices. In addition, there have been confirmed false positive cases (as high as 20%) from field validation, which can leave patients exposed to nosocomial EVD transmission, potentially limiting its application in field-tests.

Researchers have reported improved sensitivity using Fe₃O₄ magnetic nanoparticles and multifunctional nanospheres in certain applications. However, these approaches typically utilize complicated detecting agent preparations and relatively high associated diagnostic costs. Besides these well adopted clinical diagnostics methods, several bioassays have been reported in current literature, with most of the works focusing on antigen-antibody conjugation or oligonucleotide hybridization and sensing signal transduced through florescence resonance energy transfer, single-particle interferometric reflectance imaging, opto-fluidic nanoplasmonic biosensors, nanoantenna array, memristor and field-effect transistors. Some of these methods demonstrate very sensitive detection (limit of detection reaching femtomolar) attributed to delicate signal transduction mechanism. On the other hand, such high sensitivity is often at the cost of elaborate sample preparation and complicated characterization, creating challenges for low cost miniaturized POC applications.

Accordingly, there is a need for additional methods, and related aspects, of detecting Ebola virus that are low cost, sensitive, easy-to-use, and which yield rapid POC results, particularly under low resource conditions.

### SUMMARY

The present disclosure relates, in certain aspects, to ultra-sensitive gold nanoparticle (AuNP) and/or other plasmonic metal nanoparticle (MNP) colorimetric assays for portable and early-stage EBOV detection. In some embodiments, the AuNP and/or other MNP surfaces are functionalized with high affinity antibodies to EBOV related biomarkers. In these embodiments, the biomarkers generally induce the crosslinking of MNP leading to large MNP aggregate formation and precipitation, resulting in a decrease of MNP monomer concentration in solution and accordingly, a visible change in solution transparency.

In some embodiments, secreted glycoprotein (sGP) is used as a biomarker to be detected. The sGP is a disulfide linked homodimer expressed from the EBOV GP gene, taking approximately 80% of the GP gene transcription in a typical EBOV. Functioning in antigenic decoy activity during infection, sGPs are secreted in significant amounts inside host cells and released into extracellular medium, which is among the first virion related protein readily detected in patient's blood (typically at μg/ml levels). By combining the AuNP and/or other MNP solution-based assay with EVD early-stage infection biomarker sGP, some embodiments of the present disclosure provide a low cost, ultra-sensitive, colorimetric and easy-to-use EVD diagnostic assay that can detect sGP concentration down to, for example, about 1.1 μg/ml (10 nM) by naked eye or visual inspection and about 10 pg/ml (0.13 pM) in dilute human serum by portable electronic detection (e.g., UV-visible spectrometer or the like), which can be readily applied in EVD point-of-care diagnostics, especially in underdeveloped regions. These and other aspects will be apparent upon a complete review of the present disclosure, including the accompanying figures.

In one aspect, the present disclosure provides a method of detecting a virus in a sample. The method includes contacting the sample with a plurality of gold nanoparticles (AuNPs) and/or other plasmonic metal nanoparticles (MNPs) that are conjugated with one or more antibodies, or antigen binding portions thereof, that bind to an epitope of a viral antigen of or from the virus under conditions sufficient for the one or more antibodies, or the antigen binding portions thereof, to bind to the epitope of the viral antigen of or from the virus in the sample to produce bound viral antigen. The method also includes detecting the viral antigen of or from the virus when one or more aggregations of the bound viral antigen form with one another, thereby detecting the virus in the sample. Related reaction mixtures, devices, kits, and systems are also provided.

In another aspect, the present disclosure provides a method of detecting Ebola virus in a sample. In some embodiments, the method includes contacting the sample with a plurality of gold nanoparticles (AuNPs) and/or other plasmonic metal nanoparticles (MNPs) that are conjugated with at least two sets of antibodies (e.g., monoclonal antibodies or the like), or antigen binding portions thereof, in which at least a first set of antibodies, or antigen binding portions thereof, binds to a first epitope of a glycoprotein (e.g., a secreted glycoprotein (sGP), such as sGP7, sGP49, or the like) from the Ebola virus and in which at least a second set of antibodies, or antigen binding portions thereof, binds to a second epitope of a glycoprotein (e.g., the same glycoprotein (GP) comprising the first epitope or a different GP) from the Ebola virus under conditions sufficient for the first and second set of antibodies, or the antigen binding portions thereof, to bind to the first or second epitopes of the glycoproteins from the Ebola virus in the sample to produce bound glycoproteins. In some embodiments, the method includes contacting the sample with a plurality of gold nanoparticles (AuNPs) and/or other plasmonic metal nanoparticles (MNPs) that are conjugated with a single set of antibodies, or antigen binding portions thereof, that bind to an identical epitope from different monomers of a dimerized glycoprotein from the Ebola virus under conditions sufficient for the antibodies, or the antigen binding portions thereof, to bind to the identical epitope from the different monomers of the dimerized glycoprotein from the Ebola virus in the sample to produce bound glycoproteins. In some embodiments, the sets of antibodies, or antigen binding portions thereof, comprise nanobodies. In some embodiments, a given antibody, or antigen binding portions thereof, comprises a mass of between about 10 kDa and about 20 kDa (e.g., about 11 kDa, about 12 kDa, about 13 kDa, about 14 kDa, about 15 kDa, about 16 kDa, about 17 kDa, about 18 kDa, or about 19 kDa)). In some embodiments, a given antibody, or antigen binding portions thereof, comprises an equilibrium dissociation constant (KO on the order of between about 1 nM and about 100 nM (e.g., about 5 nM, about 10 nM, about 15 nM, about 20 nM, about 30 nM, about 40 nM, about 50 nM, about 60 nM, about 70 nM, about 80 nM, or about 90 nM)). The method also includes detecting the glycoproteins from the Ebola virus when one or more aggregations of the bound glycoproteins form with one another, thereby detecting the Ebola virus in the sample.

In some embodiments, the detection step comprises determining a change in absorbance at a resonance wavelength of the AuNPs and/or other MNPs. In some embodiments, the method includes quantifying an amount of the glycoproteins and/or the Ebola virus in the sample. In some embodiments, the method further includes centrifuging the aggregations of the bound glycoproteins prior to and/or during the detecting step. In some embodiments, the method further includes freezing the aggregations of the bound glycoproteins prior to the detecting step. In some embodiments, the method includes drop casting the aggregations of the bound glycoproteins prior to the detecting step. In some embodiments, the method includes obtaining the sample from a subject. In some embodiments, the method includes administering one or more therapies to the subject when the Ebola virus is detected in the sample. In some embodiments, the method includes detecting the Ebola virus within about 20 minutes or less of obtaining the sample from the subject. In some embodiments, the method includes repeating the method using one or more longitudinal samples obtained from the subject (e.g., to monitor a course of therapy and/or progression of the Ebola virus disease over time).

Essentially any sample type is used in performing the methods disclosed herein. In some embodiments, for example, the sample comprises blood, plasma, or serum. In some embodiments, the sample comprises saliva or sputum. In some embodiments, the sample comprises urine.

In some embodiments, the detecting step comprises measuring a colorimetric change when the one or more aggregations of the bound glycoproteins form with one another. In some embodiments, the method includes visually detecting the colorimetric change when the one or more aggregations of the bound glycoproteins form with one another. In some embodiments, the method includes detecting the colorimetric change when the one or more aggregations of the bound glycoproteins form with one another using a spectrometer. In some embodiments, a concentration of glycoproteins in the sample is about 15 nM or less. In some embodiments, a concentration of glycoproteins in the sample is about 100 pM or less. In some embodiments, the MNPs comprise a substantially spherical shape. In some embodiments, the MNPs comprise a cross-sectional dimension of between about 10 nm and about 100 nm.

In another aspect, the present disclosure provides a reaction mixture that includes a sample comprising Ebola virus, and a plurality of gold nanoparticles (AuNPs) and/or other plasmonic metal nanoparticles (MNPs) that are conjugated with at least two sets of antibodies, or antigen binding portions thereof, wherein at least a first set of antibodies, or antigen binding portions thereof, binds to a first epitope of a glycoprotein from the Ebola virus and wherein at least a second set of antibodies, or antigen binding portions thereof, binds to a second epitope of a glycoprotein from the Ebola virus in the sample. In another aspect, the present disclosure provides a reaction mixture that includes a sample comprising Ebola virus, and a plurality of gold nanoparticles (AuNPs) and/or other plasmonic metal nanoparticles (MNPs) that are conjugated with a single set of antibodies, or antigen binding portions thereof, that bind to the same epitopes from different monomers of a dimerized glycoprotein from the Ebola virus in the sample.

In another aspect, the present disclosure provides a device that includes at least one reaction chamber (e.g., a body structure comprising an array of wells or the like) or substrate comprising a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a glycoprotein from an Ebola virus and wherein at least a second set of antibodies, or antigen binding portions thereof, binds to a second epitope of a glycoprotein from the Ebola virus when the reaction chamber or substrate receives a sample that comprises the Ebola virus under conditions sufficient for the first and second set of antibodies, or the antigen binding portions thereof, to bind to the first or second epitopes of the glycoproteins from the Ebola virus in the sample to produce bound glycoproteins and one or more aggregations of the bound glycoproteins to produce a colorimetric change in the reaction chamber; or (ii) conjugated with a single set of antibodies, or antigen binding portions thereof, that bind to an identical epitope from different monomers of a dimerized glycoprotein from the Ebola virus under conditions sufficient for the antibodies, or the antigen binding portions thereof, to bind to the identical epitope from the different monomers of the dimerized glycoprotein from the Ebola virus in the sample to produce bound glycoproteins and one or more aggregations of the bound glycoproteins to produce a colorimetric change in the reaction chamber. In some embodiments, the one or more aggregations of the bound glycoproteins are drop cast in or on the reaction chamber or substrate. In some embodiments, a kit includes the device.

In another aspect, the present disclosure provides a system that includes a device, comprising at least one reaction chamber or substrate comprising a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a glycoprotein from an Ebola virus and wherein at least a second set of antibodies, or antigen binding portions thereof, binds to a second epitope of a glycoprotein from the Ebola virus when the reaction chamber receives a sample that comprises the Ebola virus under conditions sufficient for the first and second set of antibodies, or the antigen binding portions thereof, to bind to the first or second epitopes of the glycoproteins from the Ebola virus in the sample to produce bound glycoproteins; or (ii) conjugated with a single set of antibodies, or antigen binding portions thereof, that bind to an identical epitope from different monomers of a dimerized glycoprotein from the Ebola virus when the reaction chamber receives a sample that comprises the Ebola virus under conditions sufficient for the antibodies, or the antigen binding portions thereof, to bind to the identical epitope from the different monomers of the dimerized glycoprotein from the Ebola virus in the sample to produce bound glycoproteins. The system also includes an electromagnetic radiation detection apparatus positioned, or positionable, within sufficient proximity to the device to detect one or more colorimetric changes produced in or on the reaction chamber or substrate when one or more aggregations of the bound glycoproteins form with one another in or on the reaction chamber or substrate. In some embodiments, the electromagnetic radiation detection apparatus comprises a spectrometer. In some embodiments, the electromagnetic radiation detection apparatus comprises a microscope. In some embodiments, the electromagnetic radiation detection apparatus comprises a light-emitting diode (LED) that transmits light into and/or through the reaction chamber or substrate and a photodetector that detects light from the reaction chamber or substrate. In some embodiments, the system includes a device holder that is structured to hold the device, which device holder comprises at least one optical channel through which light is transmitted.

### BRIEF DESCRIPTION OF THE DRAWINGS

The accompanying drawings, which are incorporated in and constitute a part of this specification, illustrate certain embodiments, and together with the written description, serve to explain certain principles of the methods, reaction mixtures, devices, kits, and related systems disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically shows exemplary method steps of detecting Ebola virus according to some aspects disclosed herein.

FIGS. 2A-F show aspects of a sensing mechanism of an AuNP solution-based assay according to some aspects disclosed herein. (a) Schematic showing sGP mediated AuNP aggregation. (b) Schematic showing sGP49 surface functioned AuNP aggregation and precipitation induced supernatant color change. (c) Visual image of 60 nm AuNP solution-based colorimetric assay in detecting 100 nM and 10 nM sGP in 1×PBS. (d) Visual image of 60 nm AuNP solution-based colorimetric assay drop casted on a 1 mm thick glass slide in detecting sGP concentration from 100 fM to 1 μM in logarithmic scale. (e) Cryo-TEM image of 80 nm sGP49 surface functioned AuNP solution in the absence of sGP. (f) Cryo-TEM image of a typical aggregate in the precipitates of 80 nm AuNP solution mixing with 1 nM sGP in 1×PBS.

FIGS. 3A-F show aspects of the detection of sGP in 1×PBS using 80 nm AuNP solution-based colorimetric assay according to some aspects disclosed herein. (a) A lab-based UV-visible spectrometer system consisting of Horiba iHR320 spectrometer (left side of image) and Olympus BX53 microscope (right side of image). (b) Schematic of the characterization of AuNP solution-based assay using PDMS template as sample cuvette. (c) Visual image taken by smartphone of 80 nm AuNP solution samples loaded on PDMS template, 3 hours after AuNP mixing with 1 pM to 1 μM sGP in 1×PBS. Concentrations were assigned in logarithmic scale. (d) Absorbance spectra of samples shown in (c), measured by our UV-visible spectrometer system shown in (a). (e) Absorbance maximum plot for AuNP assay in detecting sGP and GP1,2 with concentration from 1 pM to 1 μM. Absorbance maximum is derived from spectra at AuNP resonance wavelength 559 nm. Reference sample (no sGP or GP1,2) is shown as 10-4 nM for comparison. (f) Time-resolved absorbance measured at 559 nm of 80 nm AuNP assay in detecting 10 nM sGP in 1×PBS. Detection time is defined as the time counted from the moment AuNP solution is mixed with sGP analyte.

FIGS. 4A-E show aspects of the UV-visible spectrometer, SEM and dark field scattering characterization of drop cast samples according to some embodiments disclosed herein. (a) Absorbance spectra of 80 nm AuNP solution-based colorimetric assay solutions (sGP concentration 1 pM to 1 μM in logarithmic scale) drop casted on a 1 mm thick glass slide, measured by Horiba iHR320 spectrometer. (b) Absorbance maximum derived from spectra in (a) at AuNP resonance wavelength 573 nm. Reference sample (no sGP) is shown as 10⁻⁴ nM for comparison. Inset figure shows the visual image of assay solutions drop casted glass slide. (c) Number count of bright spots from particle scattering in dark field scattering images taken for drop cast samples on gold film. sGP concentration is from 1 pM to 1 μM in logarithmic scale. Inset figure shows visual image of 80 nm AuNP assay solutions drop casted on 100 nm gold film deposited by thermal evaporator on silicon substrate. (d) SEM image of drop cast samples on gold film, taken by Hitachi S-4700 field emission SEM at 5 kV accelerating voltage. (e) 80 nm AuNP density derived from SEM images taken for drop cast samples on gold film.

FIGS. 5A-D show aspects of a centrifuge enhanced fast detection of sGP in 1×PBS using 80 nm AuNP solution-based colorimetric assay according to some embodiments disclosed herein. (a) Schematic showing fast AuNP crosslinking mediated by centrifugal concentration. (b) Absorbance spectra of AuNP assay in detecting sGP in 1×PBS with concentration from 1 pm to 1 μM in logarithmic scale, following centrifuge enhanced fast detection method with 20 minutes crosslinking time. (c) Absorbance maximum plot for AuNP assay in detecting sGP with concentration from 1 pM to 1 μM. Absorbance maximum is derived from spectra in (b) at AuNP resonance wavelength 559 nm. Reference sample is shown as 10⁻⁴ nM for comparison. (d) Influence of incubation time (detection time) on absorbance of 80 nm AuNP assay (measured at 559 nm) in detecting 10 nM sGP in 1×PBS. Incubation time is defined as time between centrifugal concentration and vortex resuspension of AuNP. Absorbance of reference sample is plotted for comparison. Inset shows narrowed absorbance range for better contrast.

FIGS. 6A-D show aspects of a point-of-care demonstration of detecting sGP in PBS buffer diluted fetal bovine serum (1:5) using miniaturized portable UV-visible spectrometer measurement system according to some embodiments disclosed herein. (a) POC-based UV-visible spectrometer system consisting of a laptop (right side of image), lamp source module (left side of image), Ocean Optics Flame spectrometer (right front) and alignment clamps (middle of image). The PDMS template loaded sample is placed in the gap between Oceanview signal reading waveguide (right) and light source head (left). (b) Absorbance spectra of PDMS template loaded samples measured by Ocean Optics spectrometer. sGP concentration is from 1 pM to 1 μM in logarithmic scale. (c) Absorbance spectra of samples measured by Horiba iHR320 spectrometer. (d) Absorbance maximum derived from spectra in (b) and (c) at AuNP resonance wavelength 559 nm. sGP concentration is scanned from 1 pM to 1 μM in logarithmic scale. Reference sample (no sGP) is shown as 10⁻⁴ nM for comparison.

FIG. 7A shows a schematic (left) and an actual photo image (right) of an LED-photodetector measurement system, which includes a LED circuit, a photodetector circuit and a 3D printed Eppendorf tube holder according to one exemplary embodiment. The LED and photodetector are embedded into two opposite recesses of an Eppendorf tube holder respectively. A channel is fabricated inside the Eppendorf tube holder to form optical path from the LED to the photodetector for signal measurement.

FIG. 7B are plots of electronic signals measured for assays detecting sGP in PBS buffer diluted fetal bovine serum (5-fold) using the LED-photodetector measurement system from FIG. 7A, shown in black colored points. Olympus BX53 measured absorbances for the same assays are plotted in blue color for reference.

FIGS. 8A-D schematically show an overview of Nano2RED assay development and characterizations. (a)-(c) Key steps of nanobody selection and surface function of nanobody on AuNPs. (d) Schematics showing different characterization and readout methods for understanding the assay mechanism, and colorimetric and quantitative determination of antigen concentration.

FIGS. 9A-F show aspects of the identification and characterization of antigen-specific nanobody binders. (a) The flowchart and timeline of nanobody binder identification. (b) Binding of single phage clones to antigen measured by ELISA. Biotinylated sGP proteins were immobilized on streptavidin-coated plates, respectively. BSA was used as a control. (c) SDS-PAGE analysis of purified sGP-specific binders. (d) BLI analysis of specific binders binding to sGP at different concentrations. The sGP proteins were immobilized on streptavidin biosensors (SA). Measured data were globally fitted (narrow grey lines). (e) Co-binder validation by sandwich ELISA. The first biotinylated nanobody binders (sGP 49) were immobilized on a plate, incubated with or without antigen, and then the second binders (sGP7) were detected by a horseradish peroxidase-conjugated antibody. (f) Co-binder validation by two-step binding characterization using BLI. Biotinylated sGP proteins were immobilized on SA sensors. Epitope binning was performed by first dipping into the first binder well for 750 s for saturation and then incubation with second binder.

FIGS. 10A-H show Ebola sGP sensing using mono-binder antibody sGP49 by incubation. (a-b) Cryo-TEM image of precipitates after 3-h incubation: (a) with 1 nM sGP, (b) the negative control (NC) sample (no sGP). (c) Visual images of samples loaded in microcentrifuge tubes, right after mixing and 3 hours after incubation. (d) The upper-level liquid samples loaded in PDMS well plate. (e) Extinction spectra measured from PDMS well plate. sGP concentration varies from 1 uM at the bottom to 1 pM and NC at the top. (f) Extinction peak (559 nm) values plotted against sGP and GP1,2 concentrations. (g) Optical signals (optical density at 450 nm) measured by sandwich ELISA in detection of sGP. (h) Extinction peak (559 nm) values extracted from spectroscopic measurements in detecting 10 nM sGP (black solid squares) and NC (red solid circles) at temperatures from 20° C. to 70° C. The buffer was 1×PBS in Figures a and b, and 5% FBS in Figures c to h. The sGP concentration was from 1 pM to 1 μM. NC sample was the buffer without sGP or GP1,2. The AuNPs were 80 nm in all measurements.

FIGS. 11A-K show rapid and electronic detection of sGP using mono-binder antibody sGP49 with improved sensing performance. (a) Modeling of aggregate formation by considering only AuNP monomer-oligomer interactions. (b) Time-dependent extinction calculated for 0.036 nM (black, as used in our experiments) and 1.8 nM (red, 50× concentrated) 80 nm AuNPs in detecting 10 nM sGP. (c) Schematic showing key steps in rapid detection protocol: centrifugation, AuNP aggregation through incubation, and vortex-mixing. (d) Visual image of AuNPs (80 nm, concentration 0.036 nM) after centrifugation at 3,500 rpm for 1 minute. (e) AuNPs in microcentrifuge tubes for rapid detection of 1 pM to 1 μM sGP in 5% FBS. The tubes were centrifuged at 3,500 rpm for 1 minute and vortex-mixed for 15 seconds. (f) Extinction spectra of AuNPs shown in (e). (g) Extinction peak values (559 nm) extracted from (f) and plotted against sGP concentration in rapid detection. Measurement results of incubation-based tests were plotted in red for comparison. (h) Effect of incubation time on the extinction measured for samples in detecting 10 nM sGP in 1×PBS. Inset shows narrowed extinction range for visual contrast. (i-k) Electronic detection of sGP in 5% FBS using miniaturized measurement system: (i) Schematic and (j) Visual image of electronic readout system, consisting mainly of a LED circuit, a photodiode circuit, and a 3D printed Eppendorf tube holder. (k) Voltage signals measured in detecting sGP in 5% FBS, shown as Blue Triangle and dashed line. Lab-based spectrometer-measured extinctions of the same assays are plotted in Black Square and solid line.

FIGS. 12A-O show co-binders sGP49/sGP7 for rapid sGP detection in different buffers. Left column: Optical images of microcentrifuge tubes after rapid test (centrifugation, 20 min incubation, and vortex mixing). Middle column: Optical images of PDMS well plates after rapid test. Right column: Optial sensing (extracted extinction peak values, in black) and electronic sensing (readout voltage, in blue) to detect sGP (or GP1,2) from 100 fM to 1 μM. (a-c) sGP spectrometric detection in 1×PBS buffer. (d-f). sGP spectrometric detection in 5% fetal bovine serum (FBS) buffer. (g-i) sGP detection in 5% human pooled serum (HPS) buffer with spectrometric readout (black squares, fitted to solid lines) and electronic readout (blue triangles, fitted to dash lines). (j-l) sGP spectrometric detection in 5% human whole blood (WB). (m-o) GP1,2 detection in 5% FBS buffer with spectrometric readout (black squares, fitted to solid lines) and electronic readout (blue triangles, fitted to dash lines).

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, reaction mixtures, devices, kits, and systems, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a secreted glycoprotein (sGP) of Ebola virus, e.g., a molecule in which one or more immunoglobulin chains is not full length, but which specifically binds to sGP. Examples of binding portions encompassed within the term “antigen-binding portion of an antibody include (i) a Fab fragment, a monovalent fragment consisting of the VLC, VHC, CL and CH1 domains: (ii) a F(ab′)2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody portions are obtained using conventional techniques known to those with skill in the art, and the portions are screened for utility in the same manner as are intact antibodies.

Conjugate: As used herein, “conjugate” refers to a reversible or irreversible connection between two or more substances or components. In some embodiments, for example, gold nanoparticles (AuNPs) are connected to antibodies and/or to antigen binding portions thereof. In some embodiments, AuNPs are conjugated with antibodies and/or to antigen binding portions thereof via one or more linker compounds.

Detect: As used herein, “detect,” “detecting,” or “detection” refers to an act of determining the existence or presence of one or more target analytes (e.g., a secreted glycoprotein (sGP) from an Ebola virus) and/or a pathogen (e.g., an Ebola virus) in a sample.

Epitope: As used herein, “epitope” refers to the part of an antigen (e.g., a secreted glycoprotein (sGP) from an Ebola virus) to which an antibody and/or an antigen binding portion binds.

Reaction Mixture: As used herein, “reaction mixture” refers a mixture that comprises molecules and/or reagents that can participate in and/or facilitate a given reaction or assay. A reaction mixture is referred to as complete if it contains all reagents necessary to carry out the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction or assay components.

Sample: As used herein, “sample” means anything capable of being analyzed by the methods, devices, and/or systems disclosed herein.

Subject: As used herein, “subject” refers to an animal, such as a mammalian species (e.g., human, dog, cat) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). In certain embodiments, the subject is a human. In certain embodiments, the subject is a companion animal, including, but not limited to, a dog or a cat. A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

System: As used herein, “system” in the context of analytical instrumentation refers a group of objects and/or devices that form a network for performing a desired objective.

### DETAILED DESCRIPTION

Ebola virus (EBOV) is a highly virulent pathogen, causing severe hemorrhagic fever in humans with 45-90% lethality. In certain aspects, the present disclosure provides a simple yet sensitive colorimetric assay for portable, fast and low-cost EBOV detection in serum or other sample types. In some embodiments, the assay includes gold nanoparticles (AuNPs) conjugated with a high-affinity antibody (secreted glycoprotein 49 (sGP49), 17.1 kDa, K_{D}=approximately 4.6 nM), which is specific to EBOV secreted glycoprotein (sGP) and capable of bridging AuNPs into aggregates upon sGP binding. This aggregation results in a decrease of monomer AuNPs concentration and accordingly, a visible change in solution color transparency. In some embodiments, for example, 100 pM or less sGP can be detected by simply analyzing the absorbance (e.g., 3 mm optical path) of 80 nm (e.g., resonance at 559 nm) AuNPs, presenting about an 8.8% change in absorbance at the resonance wavelength. Moreover, in some embodiments, the reduction of absorbance at the resonance wavelength shows accurate quantitative relation with sGP concentration in a large detection dynamic range from about 1 μM to about 0.13 pM (e.g., approximately 10 pg/ml), which overlaps with the sGP concentration level observed clinically in patients' blood.

In addition, in some embodiments, the assays disclosed herein can deliver accurate detection results in about 20 minutes or less by accelerating AuNP crosslinking, for example, using centrifuge concentration. Multiple characterization methods, including scanning electron microscopy (SEM) and dark field scattering imaging, among other techniques, can be applied for quantitative analysis in sGP detection with less than one nM sensitivity and accuracy. In some embodiments, the present disclosure also demonstrates the feasibility of detecting sGP in serum or other sample types with miniaturized portable UV-visible spectrometers for point-of-care EBOV detection. In some embodiments, for example, the AuNP solution-based colorimetric assays and other aspects disclosed herein provide ultra-high sensitivity, low cost and electricity free colorimetric detection, which can be readily utilized for point-of-care detection of Ebola virus in remote pandemic regions. These and other aspects will be apparent upon a complete review of the present disclosure, including the accompanying figures.

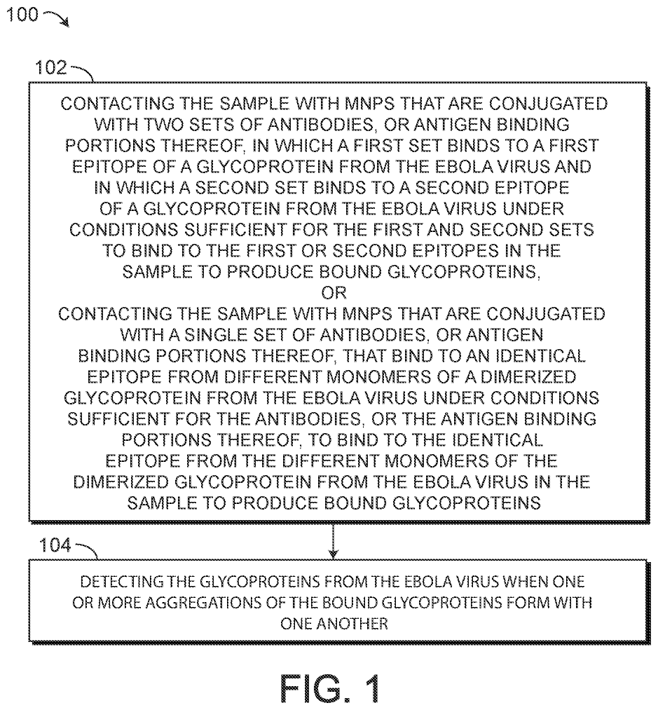

To illustrate, FIG. 1 is a flow chart that schematically shows exemplary method steps of detecting Ebola virus in a sample according to some embodiments. As shown, method 100 includes contacting the sample with a plurality of gold nanoparticles (AuNPs) and/or other plasmonic metal nanoparticles (MNPs) that are conjugated with at least two sets of antibodies, or antigen binding portions thereof, (e.g., monoclonal antibodies, nanobodies, etc.) in which at least a first set of antibodies, or antigen binding portions thereof, binds to a first epitope of a glycoprotein from the Ebola virus and in which at least a second set of antibodies, or antigen binding portions thereof, binds to a second epitope of a glycoprotein (e.g., a secreted glycoprotein (sGP)) from the Ebola virus under conditions sufficient for the first and second sets of antibodies, or the antigen binding portions thereof, to bind to the first or second epitopes of the glycoproteins from the Ebola virus in the sample to produce bound glycoproteins (step 102). In some embodiments, the plurality of MNPs are conjugated with a single set of antibodies, or antigen binding portions thereof, that bind to the same epitopes from different monomers of, for example, a dimerized glycoprotein from the Ebola virus in the sample (step 102). Essentially any sample type is used in performing method 100. In some embodiments, for example, the sample comprises blood, plasma, or serum. In some embodiments, the sample comprises saliva or sputum. In some embodiments, the sample comprises urine. In some embodiments, a given antibody comprises a mass of between about 10 kDa and about 20 kDa. In some embodiments, a given antibody comprises an equilibrium dissociation constant (K_{D}) of about 1 to about 100 nM. The production of antibodies and antigen binding portions thereof suitable for use with methods, devices, and other aspects of the present disclosure are described further herein or otherwise know to a person having ordinary skill in the art.

Method 100 also includes detecting the glycoproteins from the Ebola virus when one or more aggregations of the bound glycoproteins form with one another to thereby detect the Ebola virus in the sample (step 104). In some embodiments, the detection step includes determining a change in absorbance at a resonance wavelength of the AuNPs. In some embodiments, method 100 includes quantifying an amount of the glycoproteins and/or the Ebola virus in the sample. In some embodiments, method 100 further includes centrifuging the aggregations of the bound glycoproteins prior to and/or during the detecting step. In some embodiments, method 100 further includes freezing the aggregations of the bound glycoproteins prior to the detecting step. In some embodiments, method 100 includes drop casting the aggregations of the bound glycoproteins prior to the detecting step. In some embodiments, method 100 includes obtaining the sample from a subject. In some embodiments, method 100 includes administering one or more therapies to the subject when the Ebola virus is detected in the sample. In some embodiments, method 100 includes detecting the Ebola virus within about 20 minutes or less of obtaining the sample from the subject. The method 100 may detect the Ebola virus within 25 minutes, 24 minutes, 23 minutes, 22 minutes, 21 minutes, 20 minutes, 18 minutes, 16 minutes, 14 minutes, 12 minutes, 10 minutes, 8 minutes, 6 minutes, 5 minutes, 4 minutes, 3 minutes, 2 minutes 1 minute, or any range between these values. In some embodiments, method 100 includes repeating the method using one or more longitudinal samples obtained from the subject (e.g., to monitor a course of therapy and/or progression of the Ebola virus disease over time). In some embodiments, the detecting step comprises measuring a colorimetric change when the one or more aggregations of the bound glycoproteins form with one another. In some embodiments, method 100 includes visually detecting the colorimetric change when the one or more aggregations of the bound glycoproteins form with one another. In some embodiments, method 100 includes detecting the colorimetric change when the one or more aggregations of the bound glycoproteins form with one another using a spectrometer. In some embodiments, a concentration of glycoproteins in the sample is about 15 nM or less (e.g., when visually detecting the colorimetric change). In some embodiments, a concentration of glycoproteins in the sample is about 1 pM or less (e.g., when detecting the colorimetric change using a spectrometer). In some embodiments, the AuNPs comprise a substantially spherical shape. In some embodiments, the AuNPs comprise a cross-sectional dimension of between about 10 nm and about 100 nm.

In another aspect, the present disclosure provides a reaction mixture that includes a sample comprising Ebola virus, and a plurality of gold nanoparticles (AuNPs) that are conjugated with at least two sets of antibodies, or antigen binding portions thereof, wherein at least a first set of antibodies, or antigen binding portions thereof, binds to a first epitope of a glycoprotein from the Ebola virus and wherein at least a second set of antibodies, or antigen binding portions thereof, binds to a second epitope of a glycoprotein from the Ebola virus in the sample.

In another aspect, the present disclosure provides a device that includes at least one reaction chamber (e.g., a body structure comprising an array of wells or the like) or substrate comprising a plurality of gold nanoparticles (AuNPs) that are conjugated with at least two sets of antibodies, or antigen binding portions thereof, wherein at least a first set of antibodies, or antigen binding portions thereof, binds to a first epitope of a glycoprotein from an Ebola virus and wherein at least a second set of antibodies, or antigen binding portions thereof, binds to a second epitope of a glycoprotein from the Ebola virus when the reaction chamber or substrate receives a sample that comprises the Ebola virus under conditions sufficient for the first and second sets of antibodies, or the antigen binding portions thereof, to bind to the first or second epitopes of the glycoproteins from the Ebola virus in the sample to produce bound glycoproteins and one or more aggregations of the bound glycoproteins to produce a colorimetric change in the reaction chamber. In some embodiments, the one or more aggregations of the bound glycoproteins are drop cast in or on the reaction chamber or substrate. In other embodiments, the one or more aggregations of the bound glycoproteins may be deposited in or on the reaction chamber or substrate in a variety of deposition techniques. The deposition technique may be spin coating, dip coating, spray coating or any other similar technique known to one of skill in the art. In some embodiments, a kit includes the device.

In another aspect, the present disclosure provides a system that includes a device, comprising at least one reaction chamber or substrate comprising a plurality of gold nanoparticles (AuNPs) that are conjugated with at least two sets of antibodies, or antigen binding portions thereof, wherein at least a first set of antibodies, or antigen binding portions thereof, binds to a first epitope of a glycoprotein from an Ebola virus and wherein at least a second set of antibodies, or antigen binding portions thereof, binds to a second epitope of a glycoprotein from the Ebola virus when the reaction chamber receives a sample that comprises the Ebola virus under conditions sufficient for the first and second sets of antibodies, or the antigen binding portions thereof, to bind to the first or second epitopes of the glycoproteins from the Ebola virus in the sample to produce bound glycoproteins. The system also includes an electromagnetic radiation detection apparatus positioned, or positionable, within sufficient proximity to the device to detect one or more colorimetric changes produced in or on the reaction chamber or substrate when one or more aggregations of the bound glycoproteins form with one another in or on the reaction chamber or substrate. In some embodiments, the electromagnetic radiation detection apparatus comprises a spectrometer. In some embodiments, the electromagnetic radiation detection apparatus comprises a microscope. Exemplary devices and systems are described further herein.

### EXAMPLES

### Example 1: A Low-Cost and Simple Colorimetric Assay for High-Sensitivity Detection of Ebola Virus Glycoprotein

Results and Discussion

Sensing Mechanism

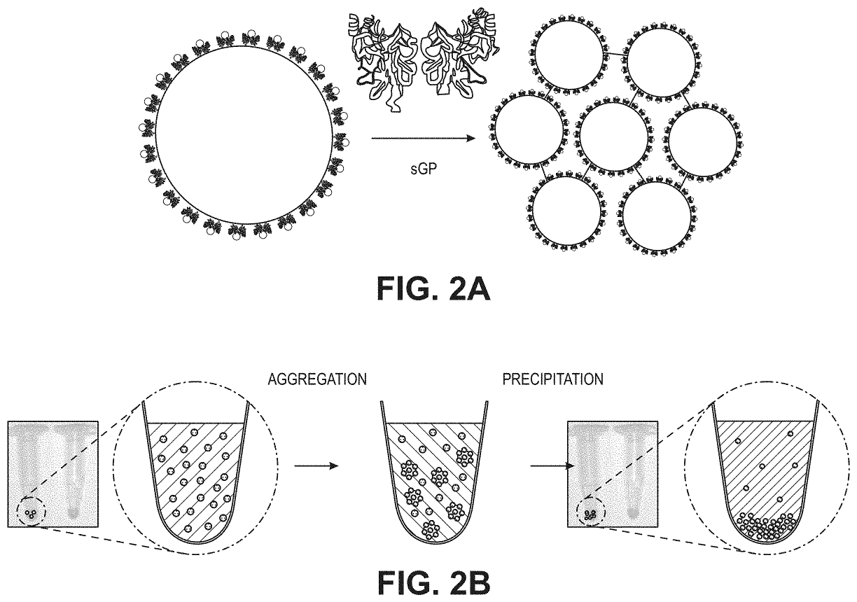

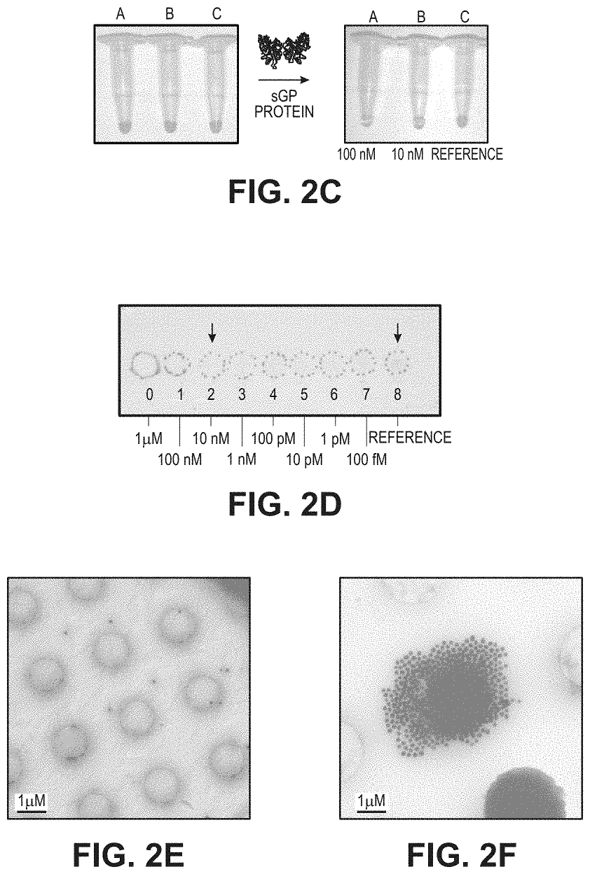

A plasmonic gold nanoparticle based assay was applied to detecting multiple species of analytes from small molecules to large cells through a variety of signal transduction mechanisms, e.g., surface plasmon resonance (SPR), surface enhance Raman spectra (SERS), AuNP enhanced fluorescent, colorimetry, scanning electron microscope (SEM) and so on. Among these mechanisms, analyte mediated crosslinking of AuNP is one of the most straightforward, cost efficient and readily characterizable signal transduction mechanisms. In some embodiments of the present disclosure, the sensing mechanism involves sGP induced crosslinking of highly specific anti-sGP monoclonal antibody sGP49 (17.1 kDa, K_{D}=˜4.6 nM) surfaced functionalized AuNP (FIG. 2A). The complete schematic of sensing mechanism is shown in FIG. 2B. Initially, all the AuNP monomers are uniformly dispersed in solution, presenting a reddish color attributed from characteristic LSPR absorbance. Upon mixing with sGP analyte at a certain concentration, AuNP monomers crosslink and form aggregates. Compared to AuNP monomers, the AuNP aggregates do not efficiently scatter light and contribute little to presenting solution color. These AuNP aggregates eventually precipitate at the bottom of the assay container by gravity force. As a result, decreased concentration of AuNP monomers in solution leads to significant color transparency change, indicating that a certain sGP concentration that is detected.

As a colorimetric sensing example, FIG. 2C shows the color change for 60 nm AuNP assay solution in detecting 100 nM (11 μg/ml) and 10 nM (1.1 μg/ml) in 1×phosphate-buffered saline (1×PBS), which are typical sGP concentrations in patients' blood. The color contrast between the AuNP assay solution detecting 100 nM and 10 nM and the reference (no sGP mixed) can be easily detected by naked eye or through analysis of images taken by smartphone, leading to portable, electricity-free point-of-care diagnostics. In addition, the assay solution can be drop cast on glass slide (1 mm thick, each drop takes 1 μL) and dried in air for visible examination, as shown in FIG. 2D. It can be observed that 100 nM and 1 nM sample spot (indexed 1 and 2) has lighter color compared the reference sample spot (no sGP, indexed 8). It is worth noting that the drop cast samples on glass slide gives the same limit of detection by naked eye down to 10 nM compared to solutions in Eppendorf tubes. In addition, drop cast of samples on glass slide shows advantage in prolonged period samples preservation. This example tested the colorimetric sensing performance for drop cast samples over 12 weeks period and the diagnostics results were accurate. This indicates that the AuNP based colorimetric assay is capable of meeting stringent POC diagnostics requirement for sample storage in underdeveloped areas where low temperature sample storage conditions are typically unavailable.

To experimentally confirm the sGP induced AuNP crosslinking and the formation of large sized aggregates at nanoscopic levels, the assay solutions were rapidly frozen and imaged by cryogenic transmission electron microscope (CryoTEM). Cryogenic sample preparation is known to prevent water crystallization and hence preserve protein structures and protein-protein interaction. Under cryogenic conditions, cryoTEM imaging can faithfully reveal the AuNP crosslinking and aggregation network in solutions. Consistent with the sensing mechanism described above, the crosslinking and aggregations of 80 nm AuNP in 10 nM sGP assay sample was confirmed in cryoTEM image FIG. 2F. The size of the aggregate in the image was approximately 2.45 by 1.92 μm. In the blank control sample, due to the lack of sGP, only 80 nm AuNP monomers were observed (FIG. 2E).

sGP Detection

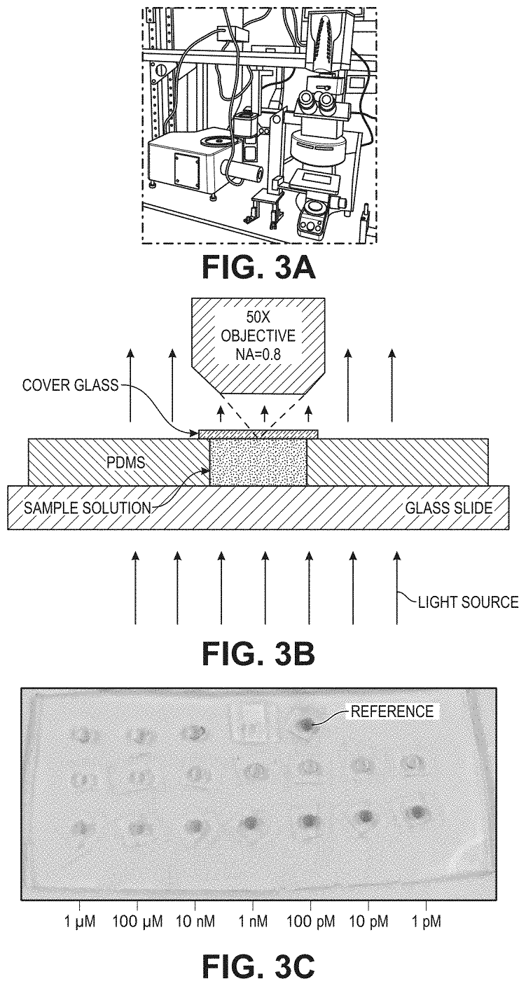

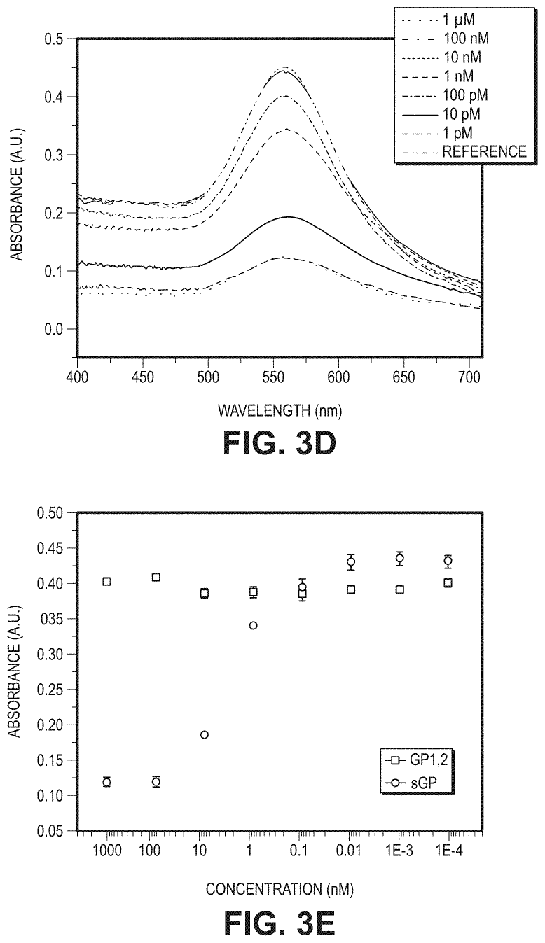

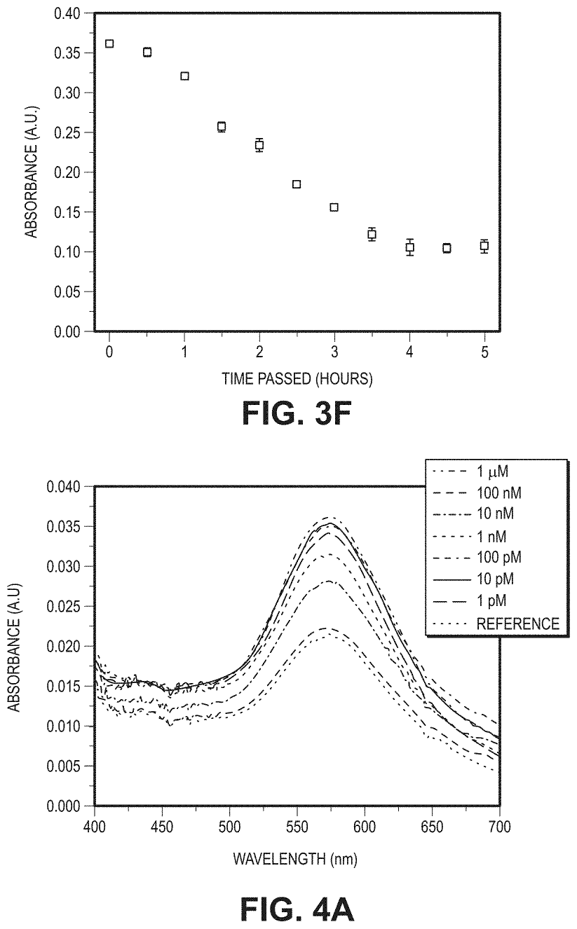

To assess the sGP detection performance of the AuNP solution-based colorimetric assay, a complete quantification of sGP detection using 80 nm diameter AuNP solution (n_{AuNP}=0.036 nM) were characterized by lab based UV-visible spectrometer with hand-made PDMS template as the sample cuvette. Here, the AuNP diameter (40 to 100 nm) was optimized and found that 80 nm AuNP delivered best sensing performance. FIG. 3A shows a lab-based UV-visible spectra measurement system consisting of Olympus BX53 microscope for signal collection and Horiba iHR320 UV-visible spectrometer for spectra measurement. As a standard measurement procedure shown in FIG. 3B, a supernatant sample (5 μL) from an Eppendorf tube contained assay solution was loaded into the 2 mm diameter hole punched through a 3 mm thick PDMS template and sealed by 100 μm thick cover glass. Then, the sample loaded PDMS was placed under 50× objective lens in an Olympus microscope system for UV-visible spectra measurement. FIG. 3C shows the image of a PDMS stamp on which 80 nm diameter AuNP solution samples mixed with sGP in 1×PBS (1 pM to 1 μM) were loaded. Visual inspection of the sample template by naked eye indicated that a gradual transparency change of solution redness color started from 1 nM to 1 μM. Significant color contrast of the assay in detecting 10 nM and higher sGP concentration to the reference sample was observed, indicating that clinically representative sGP concentrations can be readily detected by naked eyes.

To accurately characterize the optical properties of the assay solutions, UV-visible absorbance spectra of the 80 nm AuNP assay in detecting sGP in 1×PBS were measured and shown in FIG. 3D. The absorbance was defined as A=log₁₀(I₀/I), where A is the absorbance, I₀ is the intensity of light passing reference cuvette and I is the intensity of light passing sample cuvette. The measured absorbance spectra for all AuNP assay samples featured a characteristic LSPR resonance peak at 559 nm and the maximum absorbance at the peak position gradually decreased as sGP concentration increased. According to Beer-Lambert law A=εcl (A absorbance, ε extinction coefficient, c concentration, l optical path), the decrease of absorbance indicated a decreased 80 nm AuNP monomer concentration in solution, which was consistent with the sensing mechanism. The absorbance maximum was extracted at the LSPR resonance position for each assay sample and the absorbance was plotted—concentration standard curve in FIG. 3E (red datapoints). The quantification of sGP sensing shows that absorbance (A_{reference}=0.432) started to decrease at 10 pM (A_{10 pM}=0.434) and dropped quickly from 100 pM (A_{100 pM}=0.392) to 100 nM (A_{100 nM}=0.112). The absorbance stopped further decreases after sGP concentration reached 100 nM, indicating the completion of AuNP aggregation at high sGP concentration greater than 100 nM. The UV-visible spectrometer characterization of the sensors demonstrated that the 80 nm diameter AuNP solution assay achieved broadband detection dynamic range (10 pM to 100 nM) with the most sensitive linear detection region from 100 pM to 10 nM. As previously mentioned, sGP concentration found in EVD patients' blood is typically in the order of 10 nM (ug/ml). Such broadband dynamic range largely overlaps with the broadband sGP concentration in patients' blood attributed from patient-patient and infection stage variation. While early stage infection of EBOV can lead to significantly lower sGP level, which becomes very challenging to detect for ELISA and lateral flow immunoassays, the standard curve measurement showed the AuNP based colorimetric assay achieved limit of detection (LoD, defined as concentration at A_{reference}-3σ_{A}) down to 45.8 pM (5.0 ng/ml). Such broadband dynamic range and ultrahigh sensitivity indicated that the AuNP solution-based assay is ideal for quantitative sGP detection and feasible to detect early stage EBOV infection.

Specificity of detection is also a significant criterion in disease detection. Low specificity can cause false positive results leading to serious clinical incidents such as nosocomial infection. Here, the specificity of the assay was tested by detecting GP1,2, a homotrimer glycoprotein transcribed from the same GP gene as sGP. The majority of GP1,2 can be found on the virus membrane and a low concentration of GP1,2, also known as shed GP, are found in patients' blood. The close relevance of GP1,2 to sGP made it a good candidate to assess the assay's specificity. As expected, the assay showed very good specificity and no significant absorbance change at any GP1,2 concentration, indicating that no AuNP aggregates formed in the presence of GP1,2 (FIG. 3E, black datapoint set).

Next, detection speed of the sensing assay was investigated. Absorbance of 80 nm AuNP assay sensing 10 nM sGP in 1×PBS was monitored starting right after mixing sGP analyte and AuNP solution to 5 hours (FIG. 3F). The absorbance started to drop after 0.5 hour, indicating the aggregate formation happened within the half hour of mixing and precipitations started to lead the absorbance change after 1 hour. The steady aggregate formation and precipitations led to a nearly linear absorbance drop at a rate of 0.08 hr⁻¹ and the absorbance became stable after 4 hours. The dynamic absorbance test for 10 nM sGP detection indicated that the time for high contrast colorimetric detection is 2 to 3 hours. The detection speed for 40, 60 and 100 nm AuNP assay in detecting 10 nM sGP was also tested and it was found that the absorbance drop rate trend increased for bigger AuNP size, likely due to increased aggregate mass and precipitation speed.

Versatile Characterization Options for AuNP Solution-Based Assay

The assay's broad adaptability to different characterization methods not only improve its diagnostics application feasibility, diagnostics robustness and throughput but also increase the knowledge of the assay's working mechanism. In addition to UV-visible spectrometer characterization of the AuNP assay solution, the assay can be drop cast in solid form and detection can be quantified by a broad spectrum of characterization approaches such as scanning electron microscope imaging, UV-visible spectroscopy and dark field scattering imaging.

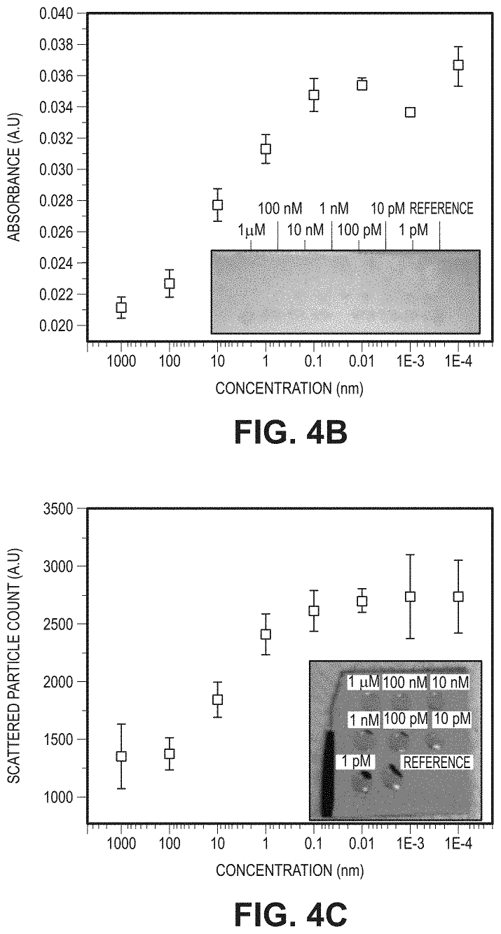

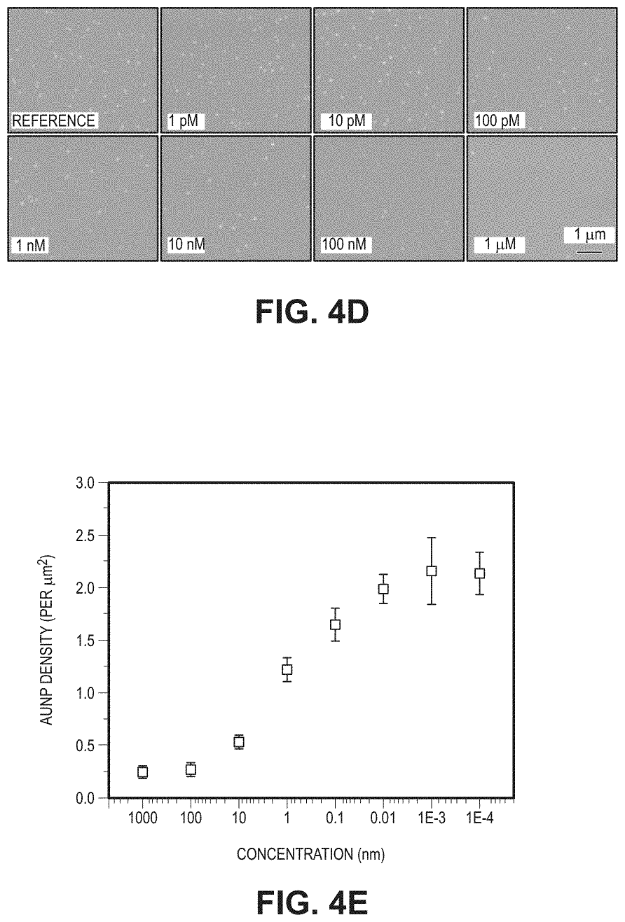

As mentioned above, the assay solution that drop cast and dried can be preserved in the absence of low temperature storage equipment such as fridge for prolonged period. Drop cast can also prevent samples from perishing in shipment from sites of sample collection to the lab-based characterization facilities. Here, the 80 nm AuNP assay solutions (n_{AuNP}=0.036 nM) detecting sGP (1 pM to 1 μM) in 1×PBS were drop cast on a 1 mm thick glass slide and their UV-visible absorbance spectra were measured (FIG. 4A). An exemplary image of dried drop cast samples is shown in the inset of FIG. 4B. The volume for each drop cast sample was 1 μL. A visual inspection by naked eye indicated the transparency of red drop cast spots started to increase at 10 nM and became readily differentiable by naked eye at 100 nM and 1 μM to reference sample. The absorbance spectra for drop cast samples featured AuNP LSPR peaks, similar to those measured from the PDMS template loaded solution samples shown in FIG. 3D. The extracted absorbance maximum standard curve for drop cast sample is plotted in FIG. 4B. Due to significantly shorter optical path (estimated ˜300 μm), the absorbance of drop cast samples is one order of magnitude smaller than that of solution samples. Similar to solution samples, the absorbance of the drop cast sample started to decrease at 10 pM (A_{10 pM}=0.035) and gradually saturated at 100 nM (A_{100 nM}=0.023) and 1 μM (A_{1 μM}=0.021). UV-visible spectra characterization of drop cast assay sample showed its effective quantitative detection of sGP down to 350 pM LoD with broad detection dynamic range (100 pM to 1 μM), offering promising solutions to simplification of sample preparation and preservation favored in POC EVD diagnostics.

In recent years, miniaturization of high-end scanning electron microscopy has made significant progress such that direct SEM image-based diagnostics of disease has become feasible. In light of recent SEM image-based diagnostics works, the 80 nm AuNP assay solutions (n_{AuNP}=0.036 nM) detecting sGP (1 pM to 1 μM) in 1×PBS was drop cast on an oxygen plasma treated gold surface and imaged by SEM. The visual image of drop cast samples on gold surface is shown in inset of FIG. 4C. Each AuNP solution sample drop cast took 1 μL of supernatant solution in an Eppendorf tube. A gold surface was selected due to its high electron conductivity that dramatically improved the contrast and resolution of electron microscopic image. From the SEM image shown in FIG. 4D, the 80 nm AuNPs were clearly recognizable for each drop cast sample. It can be observed that the density of 80 nm AuNPs gradually drops as the sGP concentration increases. It is worth noting that all AuNPs were dispersed as monomers in all drop cast samples and no large aggregates were observed since the drop cast samples were pipetted from the supernatant of assay solutions, which shows consistency to the sensing mechanisms described herein. The density of AuNP monomers was statistically measured from ten SEM images (total area 10×8.446×5.913 μm²) taken for each sGP concentration and plotted in FIG. 4E. The density of AuNPs started to decrease at 10 pM (1.97 μm⁻²) until 100 nM (0.26 μm⁻²) and saturated at 1 μM (0.24 μm⁻²), which was in accordance with UV-visible absorbance spectra measurements of both solution samples and glass slide drop cast samples. The limit of detection from SEM characterization (147 pM) was comparable to drop cast sample absorbance characterization (350 pM) but higher than assay solution absorbance characterization (45.8 pM), possibly due to increased variance in nanoscale level characterization and statistical analysis of limited sampling data.

In addition to SEM imaging, the drop cast samples on gold surfaces were characterized by dark field scattering imaging, which has been widely applied in biomolecule sensing and environmental toxin detection. LSPR mediated scattering of incident light from AuNPs on gold surface can directly indicate the density of AuNP based on the density of bright spots shown on dark field scattering image. The area of each image is 62.5×62.5 μm². It can be observed that the density of bright spots drops as sGP concentration increases, indicating the decreased concentration of AuNP in solution. The number of bright spots counted (averaged on 10 images) in each concentration is shown in FIG. 4F. The counted number of reference sample is 2753.0 and starts to drop at 10 pM (count number 2704.1) until 100 nM (counted number 1376.0) and saturates at 1 μM (count number 1348.1). Such quantitative relation between counted number and sGP concentration for dark field scattering measurement is unsurprisingly consistent with SEM image analysis and UV-visible spectra measurement. The dark field scattering measurement demonstrated its feasibility in sGP detection with sensitivity up to sub 1 nM, serving as a readily accessible characterization method for POC EVD diagnostics.

60 nm AuNP drop cast samples were also characterized by UV-visible spectrometer and 100 nm AuNP drop cast samples by UV-visible spectrometer, SEM and dark field scattering imaging. The measurement results in general show comparable sensitivity (sub 1 nM) to 80 nm AuNP.

As demonstrated above, the characterization of the AuNP solution-based assay by UV-visible spectrometer, SEM and dark field scattering imaging provide accurate quantitative sGP detection results with less than a one nM limit of detection and broad detection dynamic range. The AuNP solution-based assay offers flexible characterization options that can meet a variety of characterization requirements in different diagnostics situations.

Centrifuge Assisted Fast sGP Detection

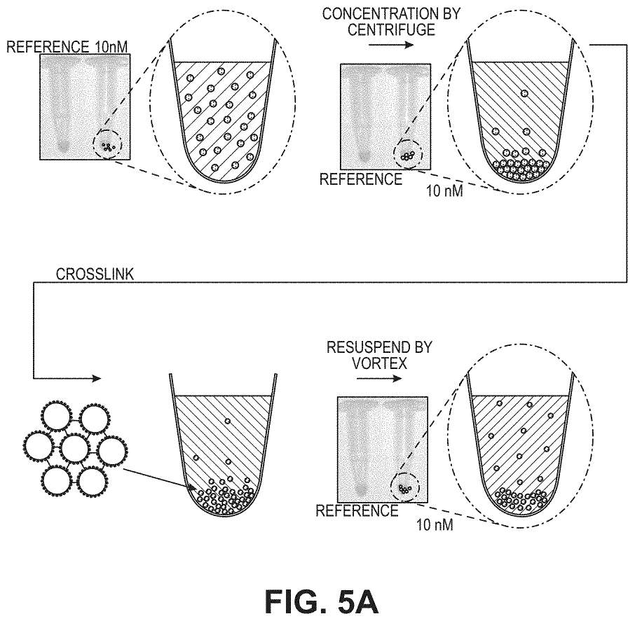

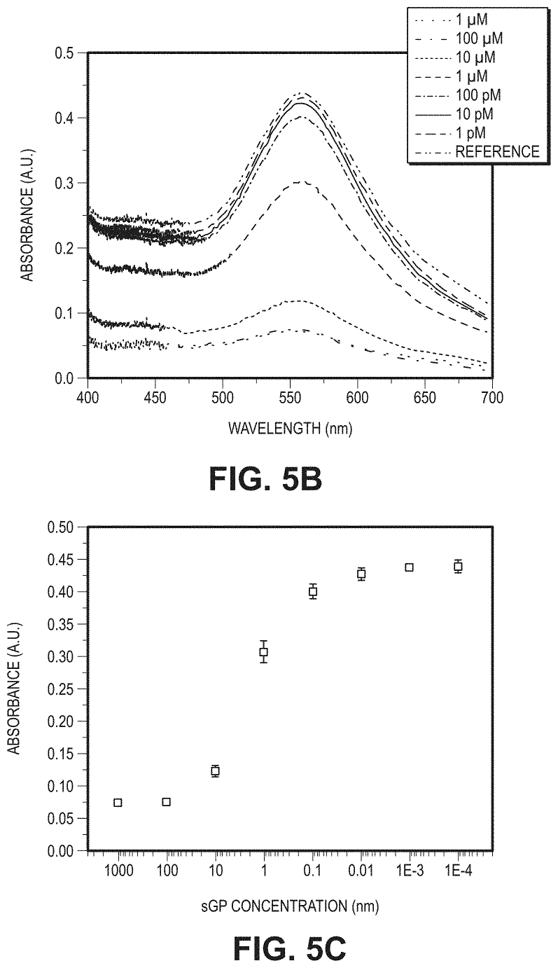

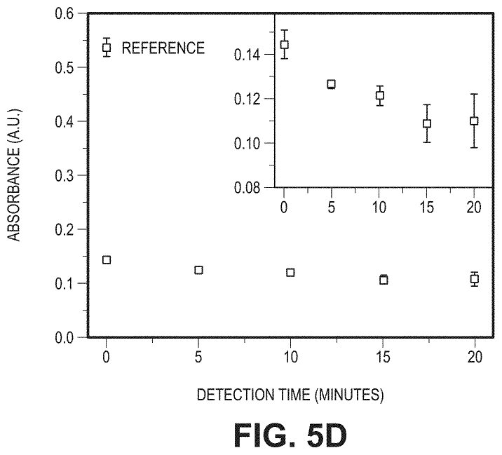

To meet the stringent requirement of fast detection in EVD in field diagnostics, the sensing mechanism was further improved and dramatically reduced the detection time from 3 hours to 20 mins using the same 80 nm AuNP assay. In the sensing mechanism mentioned above, AuNPs are uniformly dispersed in buffer solutions. According to reaction kinetics, the reaction rate is proportional to reactant concentration. Therefore, uniform dispersion of AuNP monomers at low concentration results in slow aggregate formation rate. It is straightforward to dramatically increase the AuNP monomer concentration targeted at higher aggregate formation rate. However, such approach leads to increased assay cost and consumes more analyte to reach comparable signal contrast (saturation absorbance/reference absorbance ratio). In order to simultaneously keep the same assay cost and increase the detection speed, a centrifuge assisted fast detection method is used in some embodiments. The sensing mechanism is shown in FIG. 5A. After mixing AuNP solution with sGP analyte, a centrifuge step is added to dramatically concentrate AuNP monomers at the bottom of Eppendorf tube. The centrifuge protocol is optimized at 3,500 rpm (1,200×g) for 1 minute to effectively concentrate AuNP monomers while avoiding dense AuNP aggregates formation that prevent sGP diffusion and un-crosslinked AuNP monomer resuspension. AuNP concentration is estimated to increase by at least 3 orders judging from the volume decrease (solution volume to reddish solid precipitates volume at Eppendorf tube bottom). As a result, crosslinking of AuNP at highly concentrated bottom area is greatly accelerated. After 20 minutes of AuNP crosslinking, the assay solution was thoroughly vortexed to resuspend un-crosslinked AuNP monomers back to solution. The visual image taken by smartphone confirmed the increased assay solution transparency as sGP concentration increased starting from 1 nM. The absorbance spectra of AuNP assay in detecting different concentrations of sGP in 1×PBS were measured by UV-visible spectrometer and shown in FIG. 5B. The absorbance maximum at resonance wavelength 559 nm for each sGP concentration was extracted and plotted in FIG. 5C. The absorbance-concentration standard curve for the assay undergoing centrifuge concentration was consistent with the standard curve shown in FIG. 5E, which takes three hours to yield high enough signal contrast (saturation absorbance/reference absorbance ratio: 3.60). The standard curve for centrifuge enhanced assay shows that sGP in 1×PBS starts to cause the absorbance (A_{reference}=0.439) drop at 10 pM (A_{10 pM}=0.427) and it quickly drops starting from 100 pM (A_{100 pM}=0.400) until 10 nM (A_{10 nM}=0.123) at a rate of 0.139/dec. The absorbance becomes saturated at 100 nM (A_{100 nM}=0.075) and above. Centrifuge assisted fast sGP detection assay not only showed comparable limit of detection (52.8 pM), dynamic ranges (100 nM to 10 pM) and even higher signal contrast (5.92), but tremendously reduced detection time to less than 20 minutes. Moreover, the assay utilizing centrifuge concentration method demonstrated right-after-process detection of 10 nM sGP, i.e., the color contrast is high enough that can be immediately detected by naked eye right after assay is vortexed. FIG. 5D shows the UV-visible spectrometer measured absorbance of assay solution in detecting 10 nM sGP in 1×PBS at AuNP resonance position (559 nm) with different detection time. Here detection time was defined as time for AuNP crosslinking between centrifuge and vortex step. The absorbance was 0.145 right after vortex compared to 0.536 of the reference sample. The absorbance gradually decreased to 0.110 as detection time increased to 20 minutes. It is worth noting that 10 nM and higher concentration is typical serum concentration for EVD patients. Further, right-after-process detection can lead to high screening throughput. Therefore, right-after-process detection of 10 nM sGP can be advantageous in fast screening and rapid containment in current and future EVD outbreaks.

In sum, the centrifuge assisted fast detection methods improved the AuNP assay's detection speed without inducing elaborate preparation steps and extra cost, rendering the AuNP assay feasible, for example, in field EVD diagnostics and that can be extensively applied to detection of a variety of analytes based on AuNP crosslinking.

Point-of-Care Detection of sGP in Fetal Bovine Serum

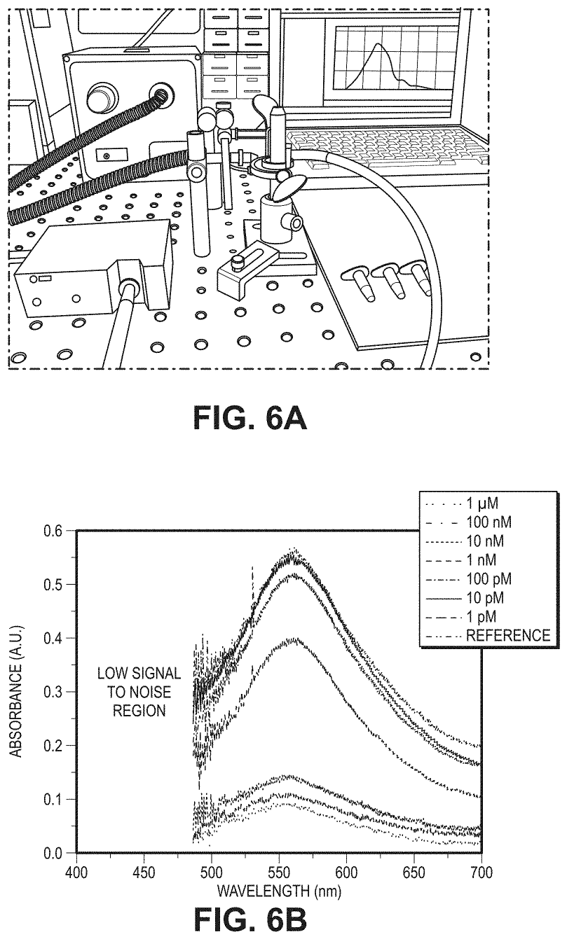

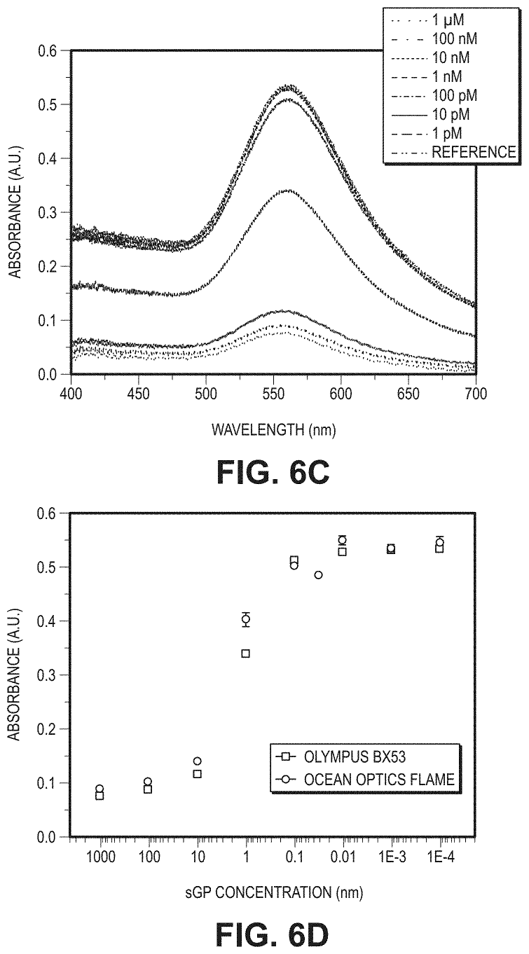

To further meet the goal of POC detection, the feasibility of detecting sGP in a fetal bovine serum sample was demonstrated using miniaturized portable UV-visible spectra measurement system towards stringent in field conditions for EBOV point of care diagnostics. The miniaturized portable UV-visible spectra measurement system consisted of a smartphone sized Ocean Optics UV-visible spectrometer (8.8×6.3×3.1 cm³), a lamp source module (15.8×13.5×13.5 cm³), alignment clamps and a laptop as shown in FIG. 6A. 80 nm AuNP solutions were mixed with fetal bovine serum containing sGP in 3:1 ratio, thoroughly vortexed and centrifuged at 1,200×g (3,500 rpm) for 1 minutes. After 20 minutes of incubation, the assay solutions were vortexed for 15 seconds and loaded into the 4 mm holes on a 3 mm thick PDMS template. The light from lamp source transmitted through the solution was collected by the waveguide head connecting to the Ocean Optics spectrometer. The absorbance spectra was measured by portable Ocean Optics spectrometer for different sGP concentrations are shown in FIG. 6B. As a comparison, the same samples were measured by lab-based Olympus BX53 microscope and Horiba iHR320 spectrometer and the absorbance spectra is shown in FIG. 6C. The absorbance maximums at the resonance wavelength 559 nm are further extracted and plotted in FIG. 6D. It can be observed that the absorbance maximum measured by Ocean Optics and Horiba spectrometer at given sGP concentration is in general consistent with each other. The difference in absorbance value is within 15.8% and possibly attributed to different collection angles in collecting the transmitted light (10× objective collection versus waveguide collecting from 4 mm aperture). In the case of serum, the absorbance measured from Ocean Optics spectrometer started to decrease at 10 pM (A_{10 pM}=0.550) and rapidly decreased from 100 pM (A_{100 pM}=0.513) to 10 nM (A_{10 nM}=0.141) at a rate of 0.186/dec. The absorbance did not saturate at higher concentrations (>10 nM) and continued to slowly decrease from 10 nM to 1 pM (A_{1 μM}=0.089) at a rate of 0.026/dec. The 52.7 pM limit of detection was comparable to that of assay detecting sGP in 1×PBS. In conclusion, it was demonstrated that the ultra-sensitive detection of sGP in serum up to 52.7 pM by using our portable UV-visible spectra measurement system. The AuNP based colorimetric assay can be readily applied to POC detection of clinical samples, showing the feasibility of low-cost, easy-to-use and ultra-sensitive quantitative POC diagnostics of EVD in underdeveloped regions.

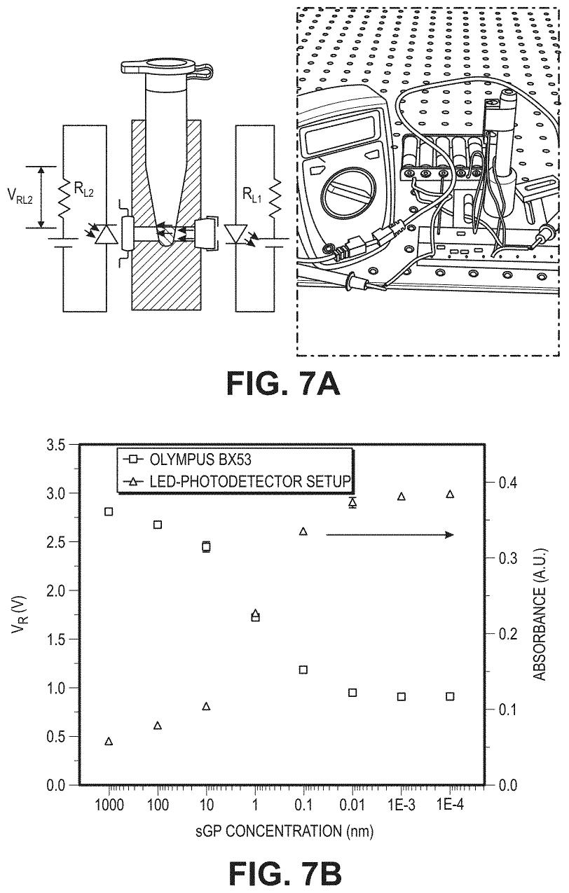

To meet an ultimate POC diagnostics objective, while the measurement system can be miniaturized it is also favorable that the system cost can be as low as possible. Here, we further simplified the measurement system by applying photodetector based electronic signal transduction mechanism, which can dramatically reduce the system cost owning to an economical commercial electronics solution. In this exemplary mechanism, the light source LED emits narrowband wavelength light only at AuNP LSPR wavelength region (λ_{P}=560 nm, FWHM_{P}=40 nm). The light transmits through the assay supernatant, strongly absorbed and scattered and subsequently measured by a photodetector which is only sensitive at a narrowband wavelength centered at AuNP absorbance maximum (λ_{S max}=560 nm, FWHM_{S}=110 nm). Finally, the light intensity is converted to photocurrent or the voltage on a serially connected load resistor that can be measured by a portable multimeter. FIG. 7A (left) shows the schematic and actual image (right) for our LED-photodetector based electronic readout system. A 3D printed Eppendorf tube holder, as small as an AA battery, firmly fixes the assay tube, LED and photodetector to their positions. A small channel is open inside the 3D printed holder to form the optical channel for signal readout. LED and photodetector are powered by AA alkaline batteries (3V and 4.5V respectively) and voltage is properly biased and divided to working points through serial resistors. Electronic readout (voltage over the photodetector load resistor) is measured by a handheld multimeter. For demonstration, we measured different concentrations of sGP in PBS diluted fetal bovine serum (five-fold) using our AuNP assay in combination with centrifuge assisted rapid detection in 20 minutes, which represents a realistic in field test situation. The electronic readings, which reflect the maximum absorbances at 559 nm for different sGP concentrations, are shown in black points in FIG. 7B. As a comparison, the same samples were characterized by lab-based microscope-coupled spectrometer (Horiba iHR320) and the absorbance maximum is plotted in blue points. It is not surprising to observe that the voltages measured show the same monotonous trend as absorbances measured by UV-visible spectrometer. The voltage level can be unambiguously differentiated from 10 pM to 1 μM, showing a broad dynamic range that is similar to referenced UV-visible spectrometer measurement results noted herein. The demonstrated quantitative relation between electronic readout and sGP concentration indicates that our LED-photodetector based electronic readout system is capable of achieving the same quantitative diagnostics results with comparable precision and accuracy as both lab-based and miniaturized UV-visible spectrometer systems. While attaining the same performance, our electronic readout system has certain advantages over spectroscopic readout systems in some respects. First, for example, the cost of the system is reduced attributable to massively manufacturable commercial components, such as LEDs and photodetectors that cost less than $1 each, as well as low cost batteries, resistors and 3D printing parts. Second, the dimension of integrated system can be scaled down to the size comparable to an 0.5 mL Eppendorf tube given the fact that miniaturized electronic components are readily available. Moreover, the electronic readout system also typically outperforms the bare eye colorimetric readout in accuracy and the results are quantitative and digitizable for data analysis. As demonstrated, we not only achieved high sensitivity and large dynamic range in rapid sGP detection, but also reduced the cost and size of assay characterization system. Our efforts on both assay and measurement system optimizations make our work a readily applicable solution for low-cost, easy-to-use, and ultrasensitive POC diagnostics of EVD in, for example, epidemic regions.

Methods and Materials

Materials

Phosphate-buffered saline (PBS) was purchase from Fisher Scientific. Bovine serum albumin (BSA) and molecular biology grade glycerol and were purchased from Sigma-Aldrich. Fetal bovine serum (FBS) was purchased from Gibco, Fisher Scientific. FBS was avoided heat inactivation to best mimic the state of serum collected in field. Poly(vinyl alcohol) (PVA, M_{W} 9,000-10,000) was purchased from Sigma-Aldrich. Sylgard 184 silicone elastomer kit was purchased from Dow Chemical. The streptavidin surface functioned gold nanoparticles were purchased from Cytodignostics, dispersed in 20% v/v glycerol and 1 wt % BSA buffer solution. Thiolated carboxyl poly(ethylene glycol) linker was self-assembled on AuNP through thiol-sulfide reaction. Streptavidin was then surface functioned through amine-carboxyl coupling mediated by N-Hydroxysuccinimide/1-Ethyl-3-(3-dimethylaminopropyl)carbodiimide (NHS/EDC) chemistry. The biotinylated sGP 49 nanobody and sGP proteins were received from collaborators in University of Washington and dispersed in 1×PBS buffer. DNase/RNase-free distilled water used in experiments and was purchased from Fisher Scientific.

Preparation of sGP49 Surface Functioned AuNP Solution

The streptavidin surface functioned gold nanoparticles (0.13 nM, 80 μL) were first mixed with excessive amount of biotinylated sGP49 nanobody (1.2 μM, 25 μL). 2 hours of incubation ensured complete streptavidin-biotin conjugation. The mixture was then purified by centrifuge (accuSpin Micro 17, Thermo Fisher) at 10,000 rpm for 10 mins and repeated twice to remove unbounded biotinylated sGP49 nanobody. The purified AuNP solution was measured by Nanodrop 2000 (Thermo Fisher) to determine the final concentration. The concentration of AuNP solution was subsequently adjusted to 0.048 nM and the was aliquoted into 12 uL in a 500 uL Eppendorf tube. High concentration sGP solution (6 μM, in 1×PBS) underwent 10-fold serial dilution and 4 uL analyte solution of each concentration (4 pM to 4 uM) was mixed with 12 uL AuNP assay and vortexed (mini vortexer, Thermo Fisher) at 800 rpm for 5 seconds. The buffer used in assay preparation and sGP dilution was prepared by diluting 10×PBS buffer and mixing with glycerol and BSA to achieve final concentration of 1×PBS, 20% v/v glycerol and 1 wt % BSA buffer.

Centrifuge Enhanced Fast Detection Method

Same procedures were followed in preparation of sGP49 surface functioned AuNP solution. After mixing sGP49 surface functioned AuNP solution with sGP analyte solution, AuNP assay solution was centrifuged (accuSpin Micro 17, Thermo Fisher) at 3,500 rpm (1,200×g) for 1 minute. AuNPs were highly concentrated the bottom of Eppendorf tube. After 20 minutes of AuNP crosslinking, the assay solution was vortexed (mini vortexer, Thermo Fisher) at 800 rpm for 15 seconds to thoroughly resuspend un-crosslinked AuNP into solution.

PDMS Template Fabrication

Sylgard 184 silicone elastomer base (consisting of dimethyl vinyl-terminated dimethyl siloxane, dimethyl vinylated and trimethylated silica) was thoroughly mixed with curing agent (mass ratio 10:1) for 30 minutes and placed in a vacuum container for 2 hours to remove the bubbles generated during mixture. The mixture was then poured into a flat plastic container in room temperature and waited for one week until the PDMS is fully cured. The PDMS membrane was then cut to rectangle shape and holes were drilled by PDMS puncher to form sample cuvette. 2 mm diameter holes were drilled for Olympus BX53 microscope characterization using 50× objective lens. 4 mm diameter holes were drilled for Oceanview spectrometer characterization and Olympus BX53 microscope using 10× objective lens. To prevent non-specific bonding of proteins on PDMS membrane surface, the PDMS surface was pre-treated with PVA. The as prepared PDMS membrane and a rectangle shaped fused silica was first rinsed with isopropyl alcohol and oxygen plasma treated at a flow rate of 2 sccm and power of 75 W for 5 min. Immediately after oxygen plasma, the PDMS membrane was bonded to fused silica to form a PDMS template that can load assay solutions into the holes. Then the PDMS template was further oxygen plasma treated at a flow rate of 2 sccm and power of 75 W for 5 min and immediately soaked in 1% wt. PVA in water solution for 10 mins. The PDMS template was subsequently nitrogen blow dried and heated on 110° C. hotplate for 15 minutes. Finally, the PDMS template was removed from hotplate, nitrogen blow cooled and ready to use in UV-visible spectra characterization.

UV-Visible Spectra, SEM Imaging and Dark Field Scattering Imaging Characterizations

The UV-visible spectra were measured from a customized optical system. An Olympus BX53 microscope equipped with a LED light source (True Color, Olympus) was used to collect light signal from sample area. The PDMS template loaded solution samples were placed on the microscope sample stage and light transmitted through AuNP assay was collected by a 50× objective lens (NA=0.8). For drop cast samples on a glass slide, a 10× objective lens (NA=0.3) was used. The spectra were measured by a microscope-coupled UV-Vis-NIR spectrometer (iHR 320, Horiba) equipped with a CCD detector. The signal was scanned from 350 nm to 800 nm with integration time of 0.01 s and averaged for 64 times.

The SEM image was taken by Hitachi S4700 field emission scanning electron microscope at 5 keV and magnification of 15,000. For each drop cast sample with different concentrations, ten images from different area were taken. The size for the area taken in each image is 8.446 μm×5.913 μm.

The dark field scattering image was taken by Olympus BX53 microscope using an EMCCD camera (iXon Ultra, Andor). A xeon lamp (PowerArc, Horiba) was used for illumination light source. The light reflected from drop cast samples were collected by a 100×dark field lens (NA=0.9). The integration time was set to 50 ms. For each drop cast sample with different concentrations, ten images from different areas were taken. The size for the area taken in each image is 62.5 μm×62.5 μm.

UV-Visible Spectrometer Characterization of sGP in Diluted Fetal Bovine Serum

Fetal bovine serum was pre-diluted five fold by 1×PBS buffer to minimize serum matrix effect. sGP solution (6 μM, in 1×PBS) was diluted in series and added to diluted fetal bovine serum to make sGP in serum solution with concentration from 4 pM to 4 uM. 30 uL 0.048 nM sGP49 surface functioned 80 nm AuNP solution was then mixed with 10 uL sGP in serum solution and thoroughly vortexed. The mixture was then centrifuged following the protocol described in centrifuge enhanced fast detection method. The AuNP crosslinking time was 20 minutes. Next, the assay solutions were loaded into 4 mm diameter holes on a 3 mm thick PDMS template and characterized by Horiba iHR 320 UV-visible spectrometer. A 10× objective lens (NA=0.3) in Olympus BX53 microscope was used to collect the signal. The signals were averagely scanned from 350 nm to 800 nm for 64 times with integration time of 0.01 s. Next, the assay solutions were characterized by a miniaturized portable UV-visible spectra measurement system. OSL2 fiber coupled illuminator (Thorlabs) was used as light source. The light passed through the 4 mm diameter holes loaded with assay solution and coupled to Flame UV-visible miniaturized spectrometer (Ocean optics) for absorbance spectra measurement. The signals were averagely scanned from 430 nm to 1100 nm for 6 times and integrated for 5 seconds for each scan.

In the next step, a LED-photodetector measurement system was devised and demonstrated for sGP sensing with reduced equipment cost. The exemplary system includes three components, namely, an LED light source, a photodetector, and a centrifuge tube holder. The centrifuge tube holder was 3D printed using ABSplus P430 thermoplastic. An 8.6 mm diameter recess was designed to snug fit a standard 0.5 mL Eppendorf tube. A 2.8 mm diameter channel was open inside the centrifuge tube holder and aligned to the LED, assay colloid and photodetector, forming an optical path for signal measurement. The LED (597-3311-407NF, Dialight) was powered by two Duracell optimum AA batteries and a 35Ω resistor was serially connected as a voltage divider to set LED operating point. The photodetector (SFH 2270R, Osram Opto Semiconductors) was reversely biased by three Duracell optimum AA batteries and serially connected to a 7 MΩ load resistor. The photocurrent that responds to intensity of light transmitted through the assay was converted to voltage through a load resistor and measured by a portable multimeter (AstroAI AM33D).

CryoTEM Sample Preparation and Imaging

To image AuNP precipitates, the supernatant was removed from the tube and 2˜3 uL of AuNP sample solution containing AuNP precipitates were left in the tube. The tube was then vortexed thoroughly. 2 uL of samples were pipetted and coated on both sides of an oxygen plasma treated Cu grid (Electron Microscopy Sciences, C flat, hole size 1.2 μm, hole spacing 1.3 μm). The oxygen plasma treatment time was 30 seconds. The Cu grid was plunge frozen in ethane using Vitrobot plunge freezer (FEI). The blot time was set to 6 seconds. After plunging, the sample was soaked in liquid nitrogen for long-term storage. For CryoTEM imaging, FEI Tecnai F20 transmission electron microscope was used with accelerating voltage set to 200 kV. More than 20 high-resolution TEM images were taken for 1 μM, 1 nM sGP in PBS samples and reference sample. The size for the area taken in each image is 4.476 μm×4.476 μm.

### Example 2: Synthetic Nanobody-Functionalized Nanoparticles for Accelerated Development of Rapid, Accessible Detection of Viral Antigens

Introduction

Successful control of emerging infectious diseases requires accelerated development of fast, affordable, and accessible assays to be widely implemented at a high frequency. Here we present a generalizable assay platform, nanobody-functionalized nanoparticles for rapid, electronic detection (Nano2RED), demonstrated in the detection of Ebola. To efficiently generate high-quality affinity reagents, synthetic nanobody co-binders and mono-binders with high affinity, specificity, and stability were selected by phage display screening of a vastly diverse, rationally randomized combinatorial library, bacterially expressed and site-specifically conjugated to gold nanoparticles (AuNPs) as multivalent in-solution sensors. Without requiring fluorescent labelling, washing, or enzymatic amplification, these AuNPs reliably transduce antigen binding signals upon mixing into physical AuNP aggregation and sedimentation processes, displaying antigen-dependent optical extinction readily detectable by spectrometry or simple electronic circuitry. With nanobodies against an Ebola virus secreted glycoprotein (sGP) as a target, Nano2RED showed a high sensitivity (limit of detection of ˜10 pg/mL for sGP and ˜40 pg/mL for RBD in diluted human serum), a high specificity, and a large dynamic range (˜7 logs). Unlike conventional assays where slow mass transport for surface binding limits the assay time, Nano2RED features fast antigen diffusion at micrometer scale, and can be accelerated to deliver results within a few minutes. The rapid detection, low material cost (estimated <$0.01 per test), inexpensive and portable readout system (<$5 and <100 cm³), and digital data output, make Nano2RED particularly suitable for screening of patient samples with simplified operation and accelerated data transmission. Our method is widely applicable for prototyping diagnostic assays for other antigens from new emerging viruses.

In recent times, we have witnessed the emergence of many infectious viral diseases, including the highly fatal Ebola virus disease (EVD, with a fatality rate of 45% to 90%). Future emergence of Disease X, as contagious as COVID-19 and as lethal as EVD, would pose an even greater threat to humanity, and will be both difficult to prevent or predict. During disease emergence, early pathogen identification and infection isolation are important for containing disease transmission. Therefore, for effective mitigation, it is necessary to accelerate the design, development, and validation of diagnostic processes, as well as to make the diagnostic tools broadly accessible within weeks of the initial outbreak.

Current diagnostic methods rely on the detection of the genetic (or molecular), antigenic, or serological (antibody) markers. Genetic diagnostics use DNA sequencing, polymerase amplification assays, or most recently, CRISPR technologies. For example, real-time reverse transcription polymerase chain reaction (RT-PCR) tests are viewed as the gold standard for their high sensitivity; however, these tests are also costly, time-consuming, and instrument-heavy. Genetic tests also can often display false positives by picking up genetic fragments from inactive viruses. In comparison, antigen and antibody detections are complementary as they allow more rapid, affordable, and accessible detection without complex sample preparation or amplification. As such, these detection methods are viewed as suitable for surveillance and timely isolation of highly infectious individuals, particularly outside clinical settings. While antibody (e.g., IgM) detection has been used for disease diagnostics, it is less predictive and more suitable for immune response studies. In comparison, viral protein antigen tests provide a reliable field-test solution in diagnosing symptomatic patients, and may serve to screen asymptomatic contacts that may become symptomatic. In addition, since they are rapid, easy to operate, and low-cost, antigen tests can be deployed at high frequencies and large volumes for in-time surveillance, which is thought to be the most important factor in disrupting a virus transmission chain.

Current antigen diagnostics typically employ enzyme-linked immunosorbent assays (ELISA) and lateral flow immunoassays (LFIs). ELISA is the workhorse for analyzing antigens and antibodies, but it requires a multistep workflow and a series of washing steps, hours of incubation prior to readout, and a readout system dependent on substrate conversion and luminescence recording. Deployment of ELISAs in high-throughput mass screenings requires automated liquid handling systems to coordinate the complex workflow, which is not ideal for portable uses. LFIs are potentially much easier to use outside lab settings but usually have much lower sensitivity and thus poorer accuracy compared to ELISA.

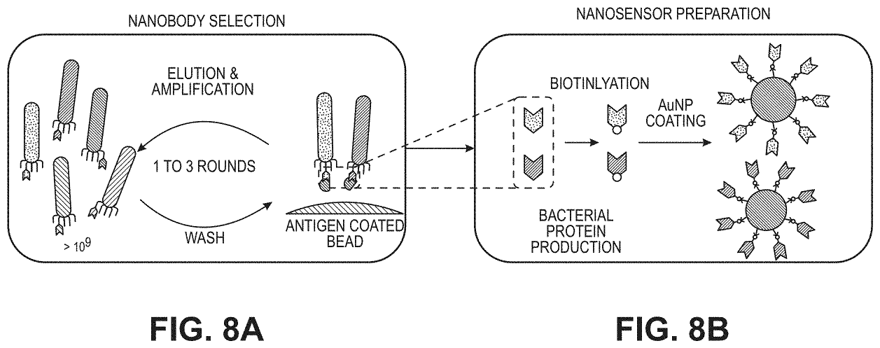

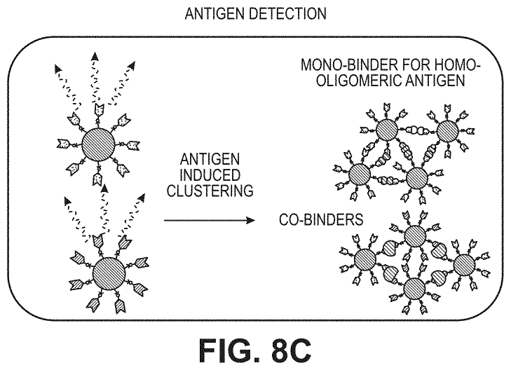

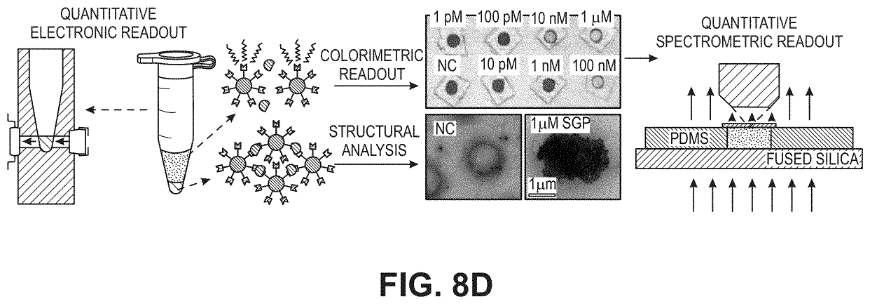

Here we report a modular strategy, i.e., nanobody-conjugated nanoparticles for rapid electronic detection (Nano2RED), which can quickly establish a rapid, accessible antigen diagnostic tool within a few weeks of pathogen identification. To generate high-quality affinity reagents within two weeks for any given purified marker protein, we have streamlined a protocol for phage selection of single-domain antibodies (or nanobodies) from a highly diverse combinatorial library (>10⁹) and bacterial expression of top hits with a significantly faster turnaround time and a lower cost than mammalian expression. Unlike traditional antibodies requiring non-specific conjugation to any solid support, potentially resulting in loss of function, nanobodies were genetically fused with an AviTag for site-specific biotinylation and immobilization onto streptavidin-coated gold nanoparticles (AuNPs). These nanobody-functionalized AuNPs serve as multivalent antigen binding sensors in our Nano2RED assays for Ebola antigen detection. Based on only physical processes, including plasmonic NP color display, NP precipitation, and semiconductor photon absorption, Nano2RED quantitatively transduces antigen binding into colorimetric, spectrometric, and electronic readouts (FIG. 8). Without fluorescent labeling or chemiluminescent readout, Nano2RED differs fundamentally from conventional high-sensitivity tests (e.g., genetic tests or ELISA) that are generally expensive and more suitable for lab use. Yet, Nano2RED greatly outperforms conventional portable and low-cost tests (e.g., LFIs), which are not qualitative or sensitive enough. Uniquely, Nano2RED features portability, low cost, and simplicity while preserving a high sensitivity (LOD of ˜0.13 pM or 11 pg/mL in Ebola sGP sensing), a high specificity (distinguishing sGP from its membrane-anchored isoform GP1,2) and a large dynamic range (˜7 logs). Additionally, its electronic readout capability can be extended to automate data collection, storage, and analysis, further reducing the workload health care workers, and speeding up diagnostic and surveillance response.

Nanobody Co-Binder Selection for AuNP Functionalization

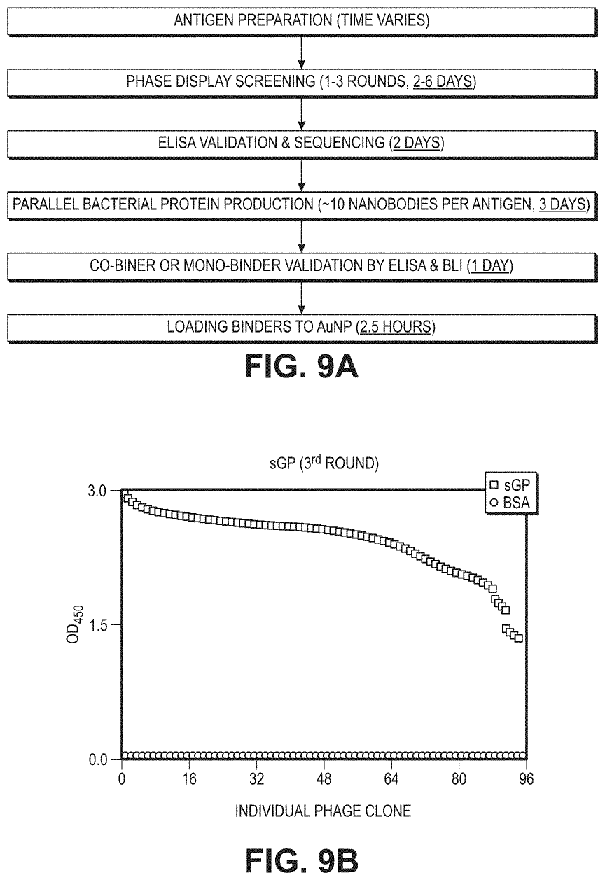

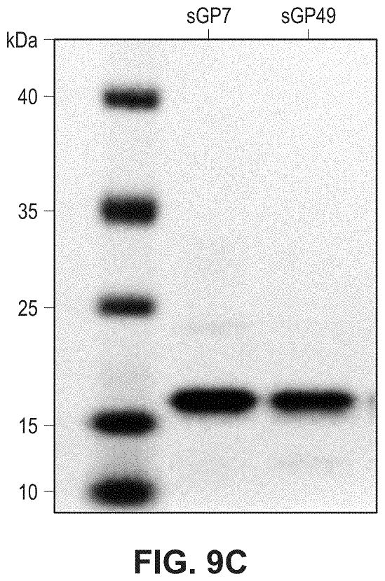

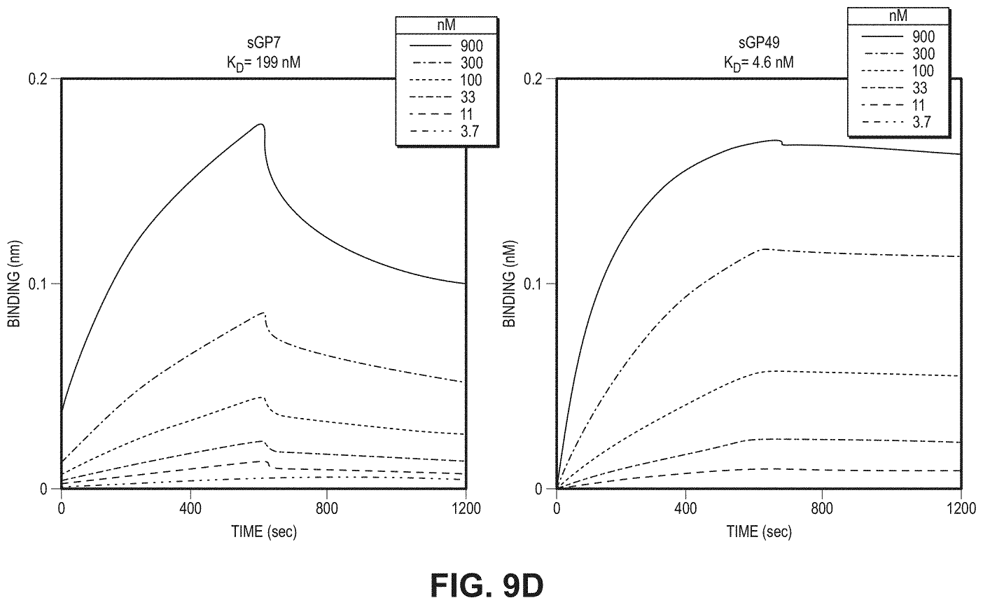

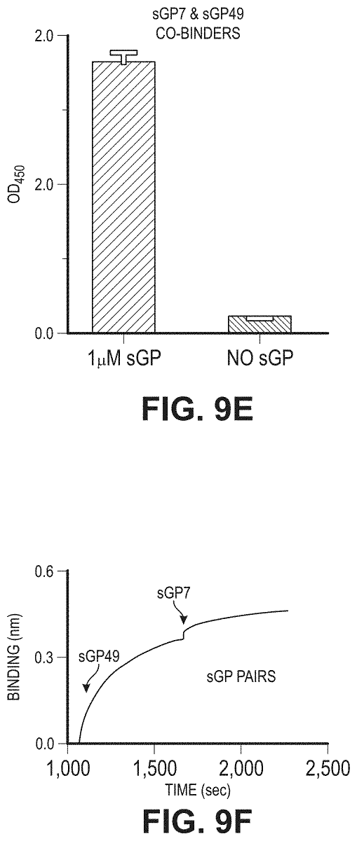

We generated nanobody co-binders (i.e., two mono-binders simultaneously binding to non-overlapping epitopes in the same antigen) against target antigens for a new in-solution assay to improve the sensitivity and specificity (FIG. 8A). Traditional methods for selecting antibody-based co-binders are slow and costly, so here we established a fast, robust protocol including the phage display selection of the combinatorial nanobody library, parallel bacterial protein production, co-binder validation, and AuNP functionalization that can be completed in less than two weeks upon the availability of an antigen protein (FIG. 9A). Nanobodies, a single-domain (12-15 kDa) functional antibody fragments from camelid comprising a universal scaffold and three variable complementarity-determining regions, are ideally suited for phage display selection and low-cost bacterial production. To avoid relatively lengthy and costly procedures and animal protection issues associated with traditional antibody screening, we screened the synthetic nanobodies library with an optimized thermostable scaffold prepared in our previous work. The Ebola antigen, sGP, is a homodimeric isoform of the glycoproteins encoded by a GP gene of all five species of Ebolavirus with multiple post-translational modifications. sGP is believed to act as a decoy to disrupt the host immune system by absorbing anti-GP antibodies. Given its abundance in the blood stream upon infection and its quantitative correlation with disease progression and humoral response, sGP is widely used as a circulating biomarker in EBOV diagnostics.

To efficiently identify nanobodies that can bind non-overlapping epitopes of an antigen protein, we assessed clonal diversity and co-binding abilities of candidates enriched in different biopanning rounds. The antigen, sGP (ManoRiver), was expressed as AviTag fusions in HEK293 and biotinylated for immobilization on streptavidin-coated magnetic beads. We identified three co-binder pairs from 10 unique nanobodies out of 96 randomly picked clones that specifically bind to sGP after three rounds of biopanning. The top co-binder pair, termed sGP7-sGP49 was bacterially expressed and purified (FIG. 9C) with high yields (1.5 to 6 mg per liter of culture). Their equilibrium dissociation constants (K_{D}) were measured to be in the nanomolar range by Bio-Layer Interferometry (BLI) (FIG. 9D) and the co-binding activities were validated by ELISA (FIG. 9E) and BLI (FIG. 9F). Lastly, nanobodies were biotinylated with E. coli biotin ligase (BirA) as previously reported and then loaded to streptavidin-coated AuNPs (see Methods section).

Nanobody-Functionalized Nanoparticles for Sensing

In our assay design, AuNPs densely coated with biotinylated nanobodies allow multivalent antigen sensing (FIG. 8A) known to significantly enhance antigen binding compared to the monovalent binding. Further, the multivalent binding also facilitates AuNP aggregation at the presence of the antigen and subsequent precipitation, producing antigen-concentration-dependent signals within minutes. The AuNP aggregation is further quantified by optical and electronic measures. In our sensing scheme (FIG. 8B), AuNPs, without nonspecific particle-particle interaction, are initially homogenously dispersed in colloid, presenting a reddish color from characteristic localized surface plasmon resonance (LSPR) extinction. Upon mixing with viral antigens, multiple AuNPs are pulled together by the antigen-nanobody binding to gradually form large aggregates. Compared to a single AuNP, the formation of AuNP aggregates gradually shifts LSPR extinction to higher wavelengths with broadened resonance attributed to plasmonic coupling between AuNPs, a phenomenon that can be simulated by finite-difference time-domain (FDTD) method. This leads to increased transparency of the AuNP colloid preciously described in DNA and protein sensing applications. Large AuNP aggregates can form pellets as gravity overtakes the fluidic drag force (FIGS. 8C and D). As a result, decreased AuNP concentrations in the upper liquid result in a colorimetric change correlated with sGP concentrations (FIG. 8D). The color change can be directly visualized by eye, and quantified in a well plate by spectrophotometer or using a simple electronic device that measures the AuNP extinction.

Finite-Difference Time-Domain (FDTD) Simulation of AuNP Extinction

We performed FDTD simulation of different numbers of AuNPs in a cluster. AuNP cluster with given number gold nanoparticles were modeled using densely packed 80 nm AuNP nanoparticles with a spacing of 12.8 nm (sGP49-sGP-sGP49 bridge length). The boundary condition along x-, y- and z-direction was set as perfect matched layer (PML). A total-field scattered-field (TFSF) light source (400-1000 nm) was used to calculate extinction cross section. The mesh size was set to be 5 nm in x-, y- and z-direction in the AuNP cluster region. Six monitors recording the power flux were set outside TFSF source region normal to x-, y- and z-directions. The background index was set as 1.33 to simulate the solution environment. The simulation time was set to 5000 fs and auto shut off threshold was set as 5×10⁶.

We found the resonance peaks red-shift for small clusters, but the resonance becomes less evident for even larger clusters, e.g., more than 10 AuNPs. This effect is expected to be related to inter-particle coupling. It also indicates that the experimentally observed extinction is likely mainly attributed to small clusters and AuNP monomers.

Colorimetric and Spectrometric Sensing of sGP

The size and shape of AuNPs determine the optical extinction and therefore the suspension color, hence affecting the sensitivity and assay incubation time. Here, the AuNP size effect was studied with NP diameters of 40, 60, 80, and 100 nm in sensing of Ebola sGP proteins using sGP49 nanobody in 1× phosphate buffered saline (PBS) buffer (described further herein). To standardize the measurement, the sGP signals were collected using a UV-visible spectrometer coupled to an upright microscope. We custom-designed a polydimethylsiloxane (PDMS) well plate bonded to glass slides as the sample cuvette. Top-level liquid from sGP sensing samples (5 μL) after incubation were loaded and inspected by optical imaging and spectroscopy readout. Clearly, as evidenced in the optical images, the color of the assay is redder for small NPs but greener for larger ones, which is attributed to a redshift in extinction resonance wavelengths at larger NP sizes. Additionally, a significant color contrast was observed in distinguishing 10 nM and higher sGP concentration from the reference negative control (NC) sample (with only PBS buffer but no sGP) for all AuNP sizes, indicating that sGP can be readily detected by the naked eye. Such colorimetric diagnostics would be very useful for qualitative or semi-quantitative diagnostics in resource-limited settings, but less ideal for quantitative and ultrasensitive detection.

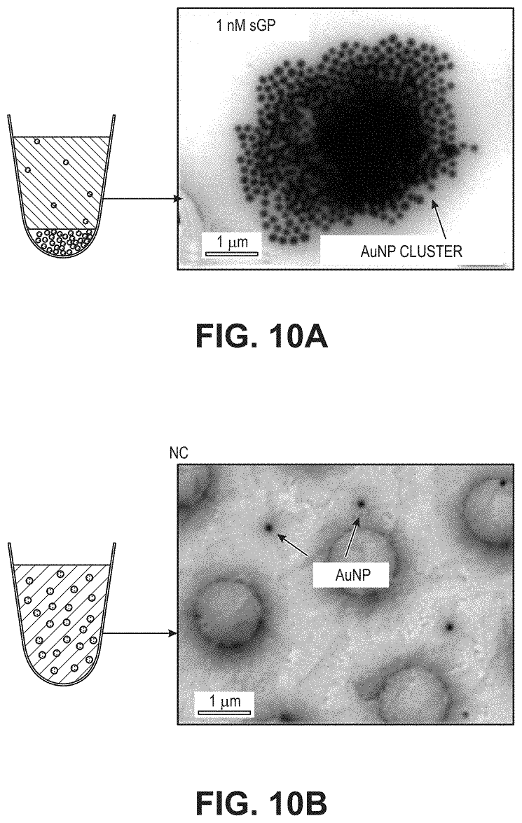

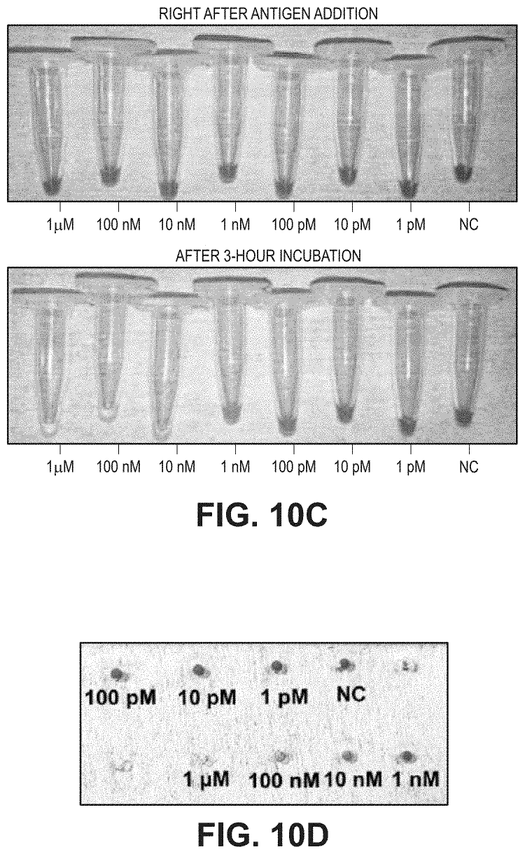

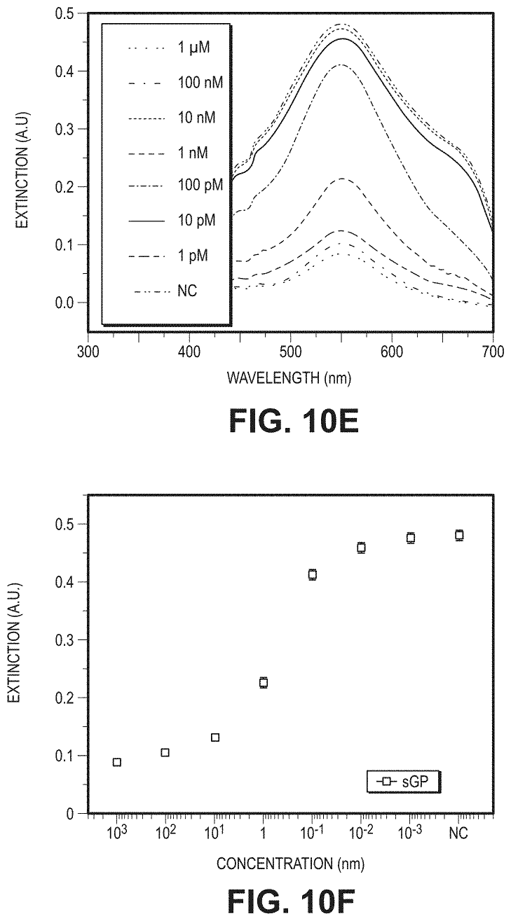

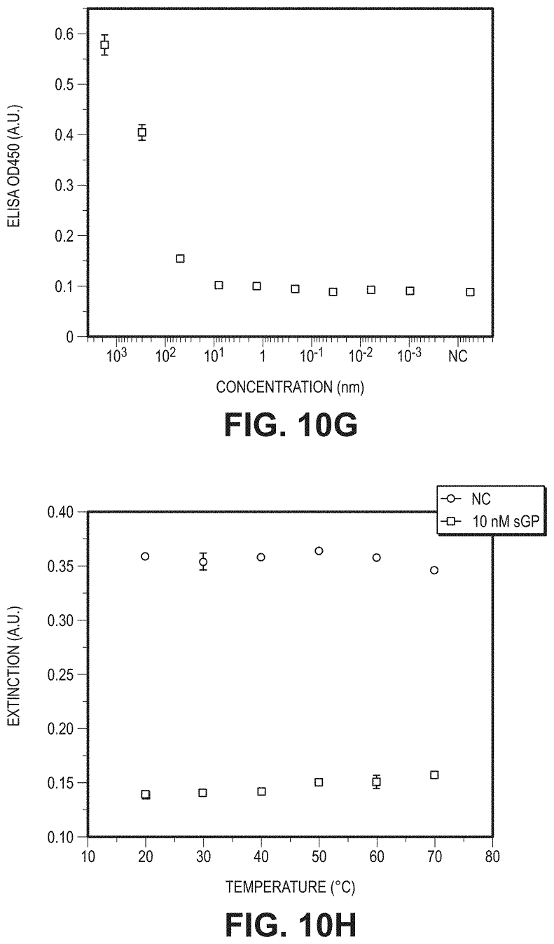

Additional accurate sGP detection was performed by quantifying the AuNP extinction signals in the PDMS plate using our spectroscopic system. The AuNP extinction is correlated with its concentration [NP] and diameter d following σₑₓₜ ∝ [NP]d³. A decrease of extinction indicated a drop of [NP] in the upper liquid level caused by antigen-induced AuNP precipitation. Further, the AuNP extinction peak values were extracted and plotted as standard curves against the sGP concentration at each AuNP size. This incubation-based assay had a large dynamic range of ˜100 pM to ˜100 nM for all AuNP sizes. In addition, the incubation was found to take 4 to 7 hours, using 40 to 100 nm NPs for detecting 10 nM sGP in 1×PBS. This NP size-dependent response could be understood intuitively from the antigen binding dynamics and the AuNP precipitation process. On the one hand, smaller NPs had higher starting concentrations, given that in our design the starting suspension extinction σₑₓₜ∝ [NP]d³ was about the same for all sizes, and therefore were expected to initiate the antigen-binding and NP aggregation reaction relatively faster. On the other hand, the precipitation of smaller aggregates took a longer time, resulting in a longer incubation period. From these experimental analyses in 1×PBS buffer, we chose 80 nm AuNPs to further characterize the assay performance in sGP sensing (FIG. 10A). This selection was based on several factors: their slightly higher sensitivity (˜15 pM, compared to ˜100 pM for other sizes), larger detection dynamic range (up to 4 logs, compared to 2 to 3 logs for other sizes), and shorter incubation time (3 to 4 hours, compared to 4 to 7 hours for other sizes).

To understand the assay's working mechanism, we complemented the solution-phase optical testing by inspecting the AuNP precipitates in solid state using different structural and optical characterization methods (described further herein). First, cryogenic transmission electron microscope (CryoTEM) images showed aggregates of AuNPs formed with 1 nM sGP with an average cluster size of 1.8 by 1.4 μm, while only 80 nm AuNP without clusters were observed in the upper-level liquid (FIG. 10B) or in the precipitates of the NC sample. This supports our sensing mechanism in that AuNP precipitation serves to transduce antigen binding to solution color change for sensing readout (FIG. 8). Further, we have performed drop-casting to deposit AuNP upper-level liquid samples on glass slides for optical extinction analysis and on gold films for scanning electron microscopy (SEM) and dark field scattering imaging. The measurement results were, in general, consistent with spectrometric in-solution sGP detection using PDMS well plate, but inferior in sensitivity (150 pM for SEM, ˜174 pM for drop-cast on glass slide, and ˜1 nM for dark filed imaging, Table 1). The decreased sensitivity could be attributed to inherent variations associated with sample preparation and background noise in the readout systems. These solid-state characterization methods were non-ideal for accessible and precise detection, given the low sensitivity and need of lab instrument for readout, but they provided valuable insight into the nanoscale NP aggregation process.

TABLE 1
Performance of the Nano2RED in Ebola sGP protein sensing

| | | | Readout | Readout | | | | | Diagnostic | |
|---|---|---|---|---|---|---|---|---|---|---|
| Assay | Readout | Readout | system | system | Assay | | Nano- | | sensitivity | |
| format | method | system | cost | size | time | | body | Buffer | pM | pg/mL |
| Incubation | Spectrometric | Lab | ~$25,000 | ~3 m³ | 3-4 | h | sGP49 | FBS | 15 | 1250 |
| | Spectrometric | microscope | | | 3-4 | h | | PBS | 174 | 14529 |
| | (cast on glass) | | | | | | | | | |
| | Dark Field | | | | 3-4 | h | | | 1000 | 83500 |
| | (cast on gold) | | | | | | | | | |
| | SEM (cast on | SEM | ~$65,000 | 3-5 m³ | 3-4 | h | | | 150 | 12530 |
| | gold) | | | | | | | | | |
| Centrifuge- | Colorimetric | Eyes or | ~$200 (phone) | ~14 × 7 × | 5-20 | min | sGP49 | FBS | 1000 | 83500 |
| accelerated | | smart phones | | 0.8 = 78 | | | sGP49/ | FBS | 100 | 8350 |
| (rapid) | | | | cm³ | | | sGP7 | | | |
| | Spectrometric | Lab | ~$25,000 | ~3 m³ = | | | sGP49 | FBS | 80 | 6680 |
| | | microscope | | 3,000,000 | | | sGP49/ | PBS | 0.16 | 13 |
| | | | | cm³ | | | sGP7 | FBS | 1.05 | 88 |
| | | | | | | | | HPS | 1.26 | 105 |
| | | | | | | | | WB | 18.2 | 1520 |
| | | Portable | $1,000 | 8.8 × 6.3 × | | | sGP49 | FBS | 99 | 8270 |
| | | spectrometer | | 3.1 = 170 | | | | | | |
| | | | | cm³ | | | | | | |
| | Electronic | LED & | ~$5 | 1.1 × 1.1 × | | | sGP49 | FBS | 27 | 2250 |
| | | photodetector | | 3.3 = 4 | | | sGP49/ | HPS | 0.13 | 11 |
| | | | | cm³ | | | sGP7 | | | |

sGP was further detected in diluted fetal bovine serum (FBS, 5%) using 80 nm sGP49-functionalized AuNPs. Similarly, to test in 1×PBS, after 3-hour incubation in microcentrifuge tubes (FIG. 10C), the upper-level liquid samples were loaded into a PDMS well plate (FIG. 10D) and measured by spectrometer (FIG. 10E). From the plot of extinction peak values against sGP concentration (FIG. 10F), our assay could again detect sGP over a broad range from 10 pM to 100 nM, which supports clinically relevant Ebola detection from patients' blood (sub-nM to μM). Here the three-sigma limit of detection (LoD), defined as the concentration displaying an extinction differentiable from the NC sample (E_{NC}), or E_{NC}−3σ where σ is the measurement variation of all samples, was found to be about 15 pM (or 1.25 ng/mL), comparable to that measured using sGP49 phage ELISA (LOD estimated ˜80 pM, FIG. 10G and Table 2). The LOD can be understood from simple and rough estimations based on the nature of multivalent antigen binding (described further herein). We also found the 10 nM sGP could be easily distinguished from NC sample at a broad temperature range from 20 to 70° C. (FIG. 10H). This indicates our assay can be transported, stored, and tested at ambient temperatures without serious concerns of performance degradation, which is very important for mass screening.

TABLE 2
LOD analysis of Ebola sGP detection with Phage-ELISA

| | | | | σ (A.U.) | | | LOD (pM) | | |
|---|---|---|---|---|---|---|---|---|---|
| Antigen | Binder | Media | Readout method | σ_{PS4} | σ_{PSA} | σ_{NC} | σ_{PS4} | σ_{PSA} | σ_{NC} |
| Ebola | sGP49 | PBS | Colorimetric | 0.00121 | 0.00851 | 0.000622 | 76 | 6200 | 38 |
| sGP | | | | | | | | | |

Impact of Nanoparticle Size: sGP Sensing by Incubation

Here, the AuNP size effect was studied with NP diameters of 40, 60, 80, and 100 nm in sensing of Ebola sGP proteins from 1 pM to 1 μM in 1×PBS buffer. The sGP signals were collected using a UV-visible spectrometer coupled to an upright microscope. We custom designed a polydimethylsiloxane (PDMS) well plate, consisting of 2 mm diameter and 3 mm thick punched holes, that is bonded to a 0.5 mm thick diced fused silica.

Additionally, the AuNP concentrations were adjusted to have roughly identical optical density levels at their peak plasmonic resonance wavelengths (533, 544, 559, and 578 nm for 40, 60, 80, and 100 nm diameter), at an AuNP concentration [NP] of 0.275, 0.086, 0.036, and 0.019 nM, respectively. The extinction coefficient of NPs is theoretically proportional to their total mass (or volume) as σₑₓₜ ∝ [NP]d³, therefore, [NP] drops with the particle diameter given we intentionally standardize the total extinction of all the NPs.

Here the UV-visible extinction can be mathematically defined as

$$E = {log}_{10} ( \frac{{I}_{0}}{I} ) = ɛ c l ,$$

where E is the measured AuNP extinction, I₀ and I are the light intensity collected at the reference and sGP sample cuvettes, ε is the AuNP extinction coefficient, c is AuNP concentration, and l is optical path (the solution depth, ˜3 mm here).

We further extracted the extinction peak intensity for each assay sample and plotted the standard curves of extinction versus concentration for each AuNP size. The sGP signal at low concentration (<10 pM) was indistinguishable from the NC signal (with an extinction E_{NC} within the range of 0.4-0.45).

In addition to the detection limits, the assay detection time was studied at 10 nM sGP concentration in 1×PBS. The extinction generally started to drop after 0.5-1.5 hour for all sizes, indicating a stage to initiate aggregate formation and precipitation. Extended incubation led to a nearly linear extinction drop, at a rate of 0.049, 0.071, 0.080, and 0.091 hr⁻¹ for 40, 60, 80, and 100 nm NPs, eventually reaching a stable value after 7, 5.5, 4, and 4.5 hours, respectively.

Rapid Antigen Detection

The PDMS well plate-based spectrometric measurement required about 3 hours incubation for effective AuNP bridging and precipitation, which is shorter than ELISA and much better than many RT-PCR assays. However, rapid diagnostics, that is, less than 30 minutes, is more desirable for accessible infectious disease diagnosis and control of disease spread. Here, we further studied the sensing mechanism, aiming to reduce the detection time (described further herein). In conventional ELISA assays, the antigen diffusion process is usually the rate-limiting step, since the fluidic transport to a solid surface is ineffective given long diffusion length (millimeter scale liquid depth in well plate) and slow fluidic flow speed at plane surfaces (near zero surface velocity). This leads to slow mass transport and ineffective surface binding, and an accordingly long assay time. Differently, diffusion is no longer the limiting factor in our assay, given the use of NPs in lieu of plane surfaces as reaction sites. The diffusion length is estimated to be about 1 μm due to a high NP concentration (e.g. ˜0.036 nM for 80 nm AuNPs). The small size and mass of AuNPs (5×10⁻¹⁵ g) result in a high diffusivity (D_{NP}˜4.2 μm²/s from Stokes-Einstein equation) and a high thermal velocity (estimated 0.028 m/s). All of these features promote effective fluidic transport and antigen binding, with an estimated diffusion time of <1 sec.

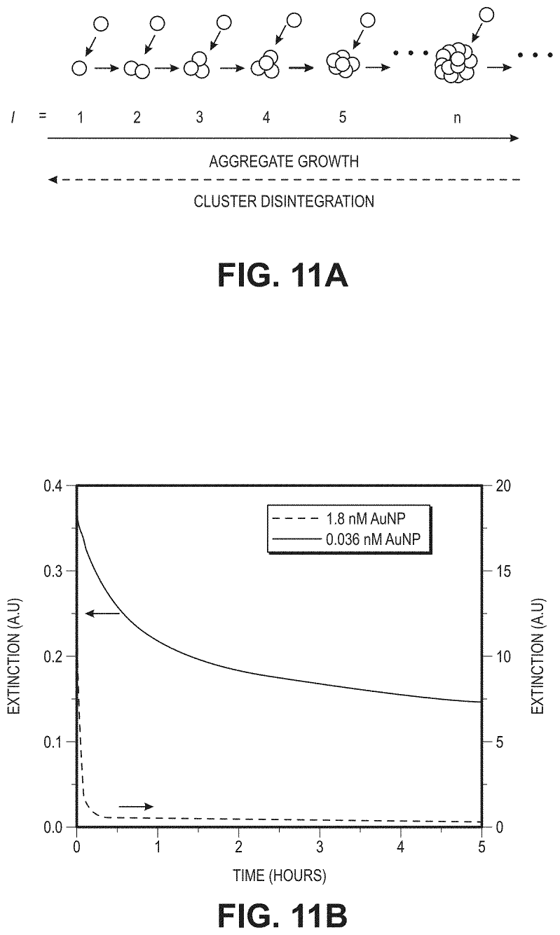

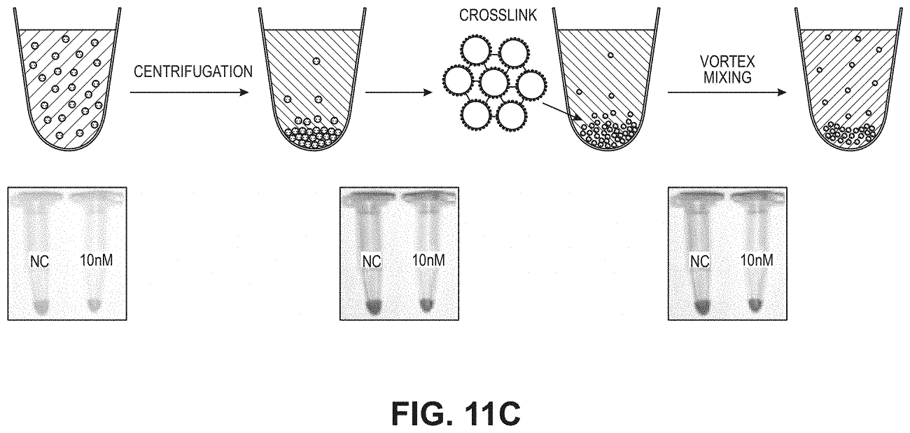

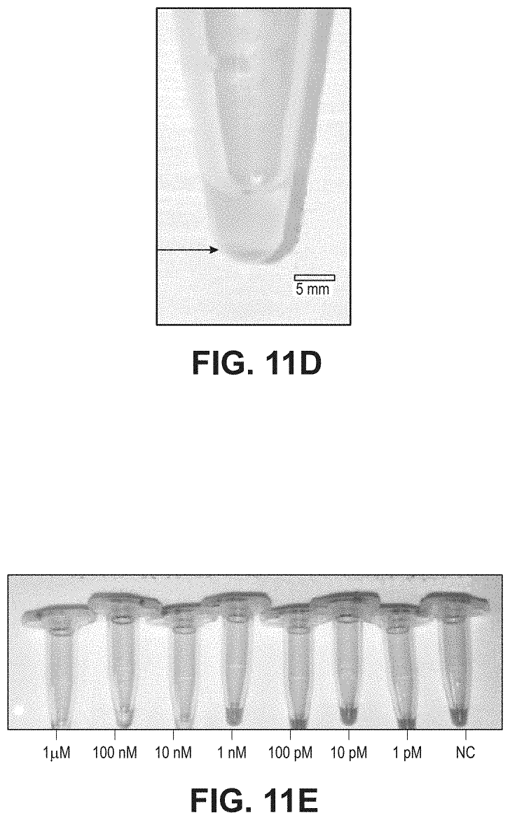

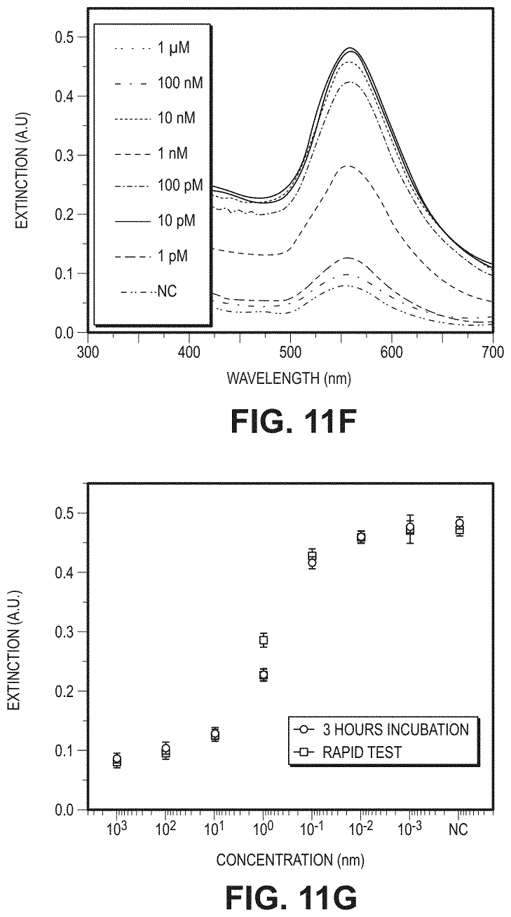

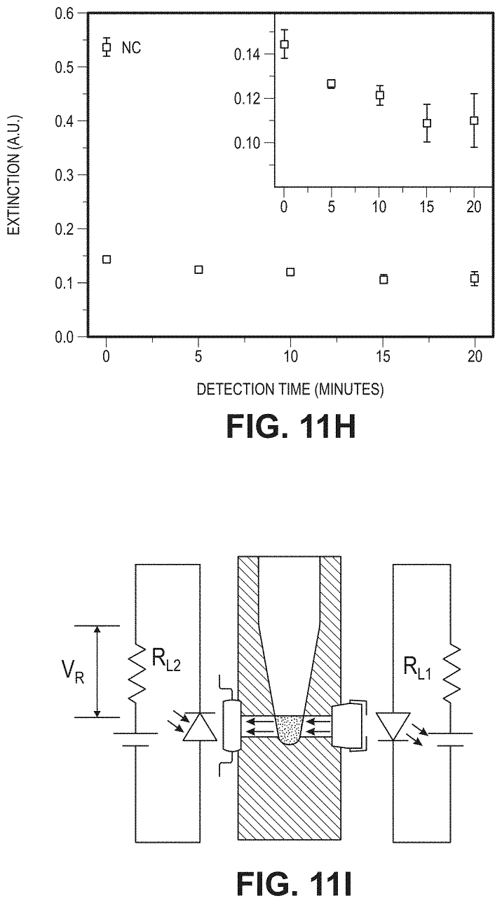

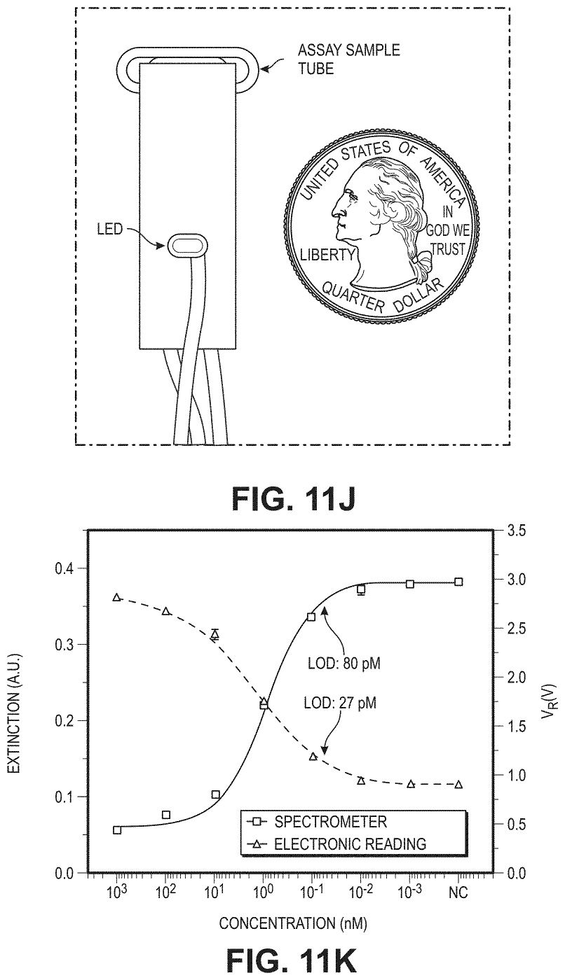

We further speculated that the AuNP aggregation and precipitation process could play important roles in determining assay time. Here, we developed a simplified model based on Smoluchowski's coagulation equation to understand the aggregation process (FIGS. 11A and B, and more details described herein). Briefly, an empirical parameter P, which defines the probability of antigen-nanobody binding per collision, has a large impact on the modelled assay time. By comparing to experimentally measured assay incubation time, we found using P=1, that is very high-affinity binding, provides a much better prediction compared to using a smaller P value calculated by the ELISA-measured kinetic constants. This observation is attributed to two factors: the ELISA-measured binding kinetics is strongly affected by the surface-limited diffusion process and could not precisely estimate the true nanobody-antigen binding in solution; and the multivalence of the nanobody-bound AuNPs greatly improves the observed “functional affinity” compared to intrinsic mono-binding affinity. Using this model, it was estimated that the aggregation time constant τ_{agg} as 0.87 hour at 0.036 nM AuNP in detection of 10 nM sGP. Yet τ_{agg} could be greatly reduced by increasing AuNP concentration, for example to 0.024 hour, or 36 times shorter, when using 50 times more concentrated NPs.

On the other hand, as gravitational force overcomes fluidic drag, large clusters precipitate to form sedimentation and continuously deplete AuNPs and sGP proteins in the colloid until reaching equilibrium. The sedimentation time can be estimated using the Mason-Weaver equation by τ_{sed}=z/(s·g) where z is the precipitation path (for example the height of colloid liquid), g is the gravitation constant, and s is the sedimentation coefficient dependent on the physical properties of AuNPs and buffers. Given that z ˜3.5 mm for 16 μL liquid in a microcentrifuge tube, we calculated that τ_{sed} decreases from 26 hours for 80 nm AuNPs to 1.0 and 0.3 hours for a 400 nm and 800 nm diameter cluster (comparable to experimentally observed clusters of micrometer size at 1 nM sGP), respectively. The estimated aggregation and precipitation times are consistent with the experimentally observed incubation time (˜3 hours).

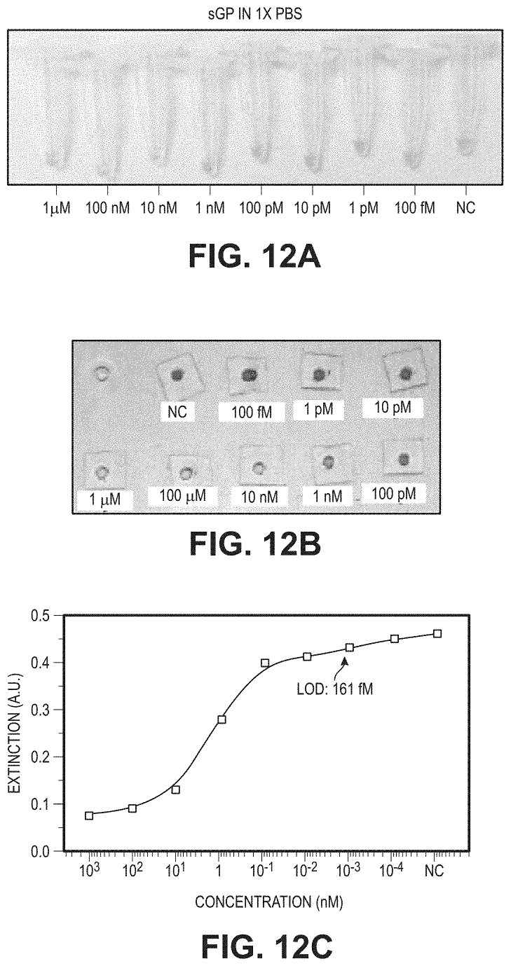

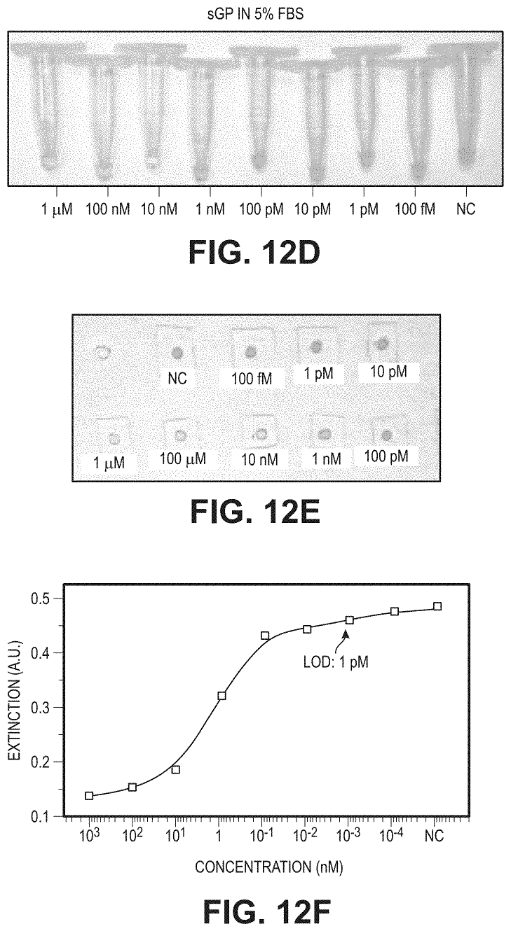

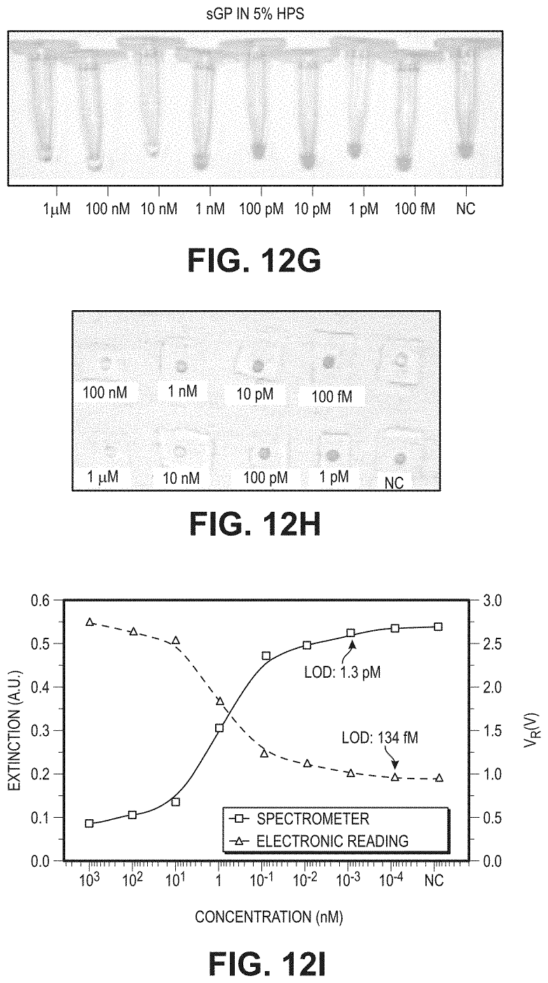

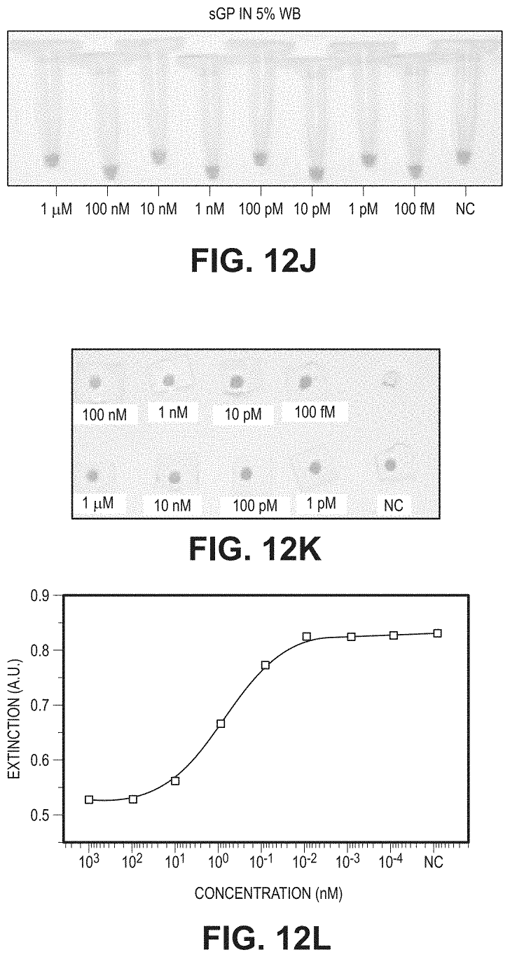

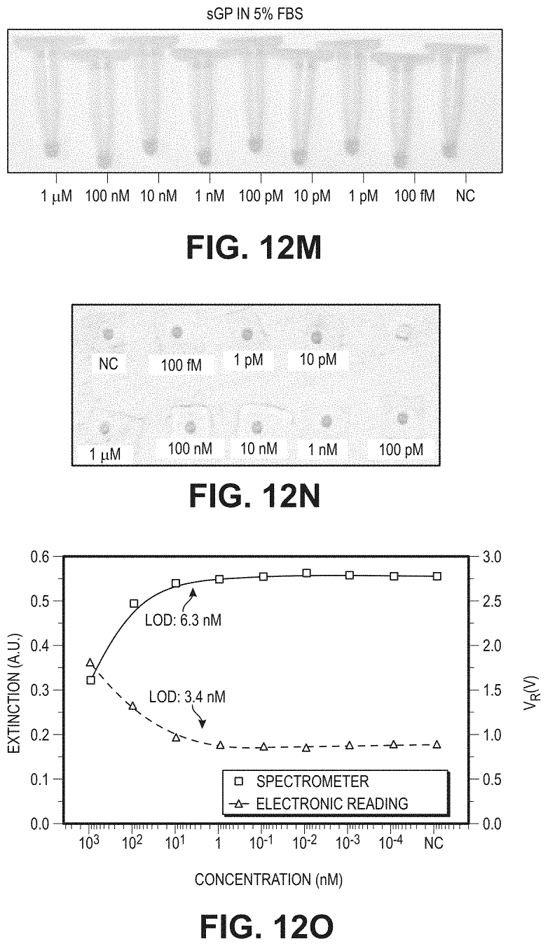

For rapid detection, we introduced a centrifugation step (1,200×g, 1 min) after antigen mixing to both enhance the reagents' concentration and decrease the precipitation path (FIG. 11C, additional data described further herein). This step concentrated AuNPs at the bottom of microcentrifugation tubes without causing non-irreversible AuNP aggregation, with an estimated z of ˜150 μm as seen from optical image (FIG. 11D). This corresponds to a roughly >20 times reduction in precipitation path and accordingly τ_{sed}. Additionally, the concentrated AuNPs are confined to an estimated <0.34 μL volume, or ˜50 times concentration increase from original 16 μL colloid liquid, leading to a greatly reduced τ_{agg}, estimated from 0.87 hour to 0.024 hour (FIG. 11B). These calculations indicate that both the aggregation formation and precipitation of the aggregates can take place in just a few minutes, important to shortening assay time. To experimentally validate the rapid detection concept, the assay colloid was incubated for 20 min after centrifugation and then thoroughly vortexed, which served to re-suspend free AuNPs that could have been physically adsorbed to the tube bottom. Indeed, the increased upper-level assay liquid transparency at higher sGP concentration (FIG. 11E) was distinguished visually for sGP >1 nM. The extinction values of the upper-level liquid (FIG. 11F) were extracted at its peak wavelength (˜559 nm) (FIG. 11G), and plotted against sGP concentration, along with the 3-hour incubation results. The rapid test presented comparable performance in dynamic range and LOD (˜80 pM) compared to incubation. Using 10 nM sGP as the antigen, we found the color contrast was high enough to be immediately resolved by the naked eye after vortex mixing, requiring minimal incubation (FIG. 12H). Including all of the operation steps for sample collection, pipetting, centrifugation, vortex mixing, and readout, this rapid test scheme can be completed in a few minutes.

Characterization of sGP Sensing Monobinders

Preparation of Mono-Binder Surface Functioned AuNP Colloid

The gold nanoparticles (0.13 nM, 80 μL) that were already surface-functioned with streptavidin were first mixed with an excessive amount of biotinylated sGP49 nanobody (1.2 μM, 25 μL). The mixture was then incubated for 2 hours to ensure complete streptavidin-biotin conjugation. Next, the mixture was purified by centrifuge (accuSpin Micro 17, Thermo Fisher) at 10,000 rpm for 10 mins and repeated twice to remove unbounded biotinylated sGP49 nanobody. The purified AuNP colloid was measured by Nanodrop 2000 (Thermo Fisher) to determine the final concentration. The concentration of AuNP in colloid was subsequently adjusted to 0.048 nM and was aliquoted into 12 uL in a 500 uL Eppendorf tube. sGP stock solution (6 μM, in 1×PBS) underwent a 10-fold serial dilution and a 4 uL sGP solution of each concentration (4 pM to 4 uM) was mixed with 12 uL AuNP assay colloid and briefly vortexed (mini vortexer, Thermo Fisher) at 800 rpm for 5 seconds. The buffer used in assay preparation and sGP dilution was prepared by diluting 10×PBS buffer and mixing with glycerol and BSA to reach a final concentration of 1×PBS, 20% v/v glycerol and 1 wt % BSA.

Tem Inspection.

The assay colloids at the bottom of Eppendorf tube were collected and imaged by cryogenic transmission electron microscope (CryoTEM). Cryogenic sample preparation is known to prevent water crystallization and hence preserve protein structures and protein-protein interaction. Clearly, aggregates of AuNPs formed with 1 μM sGP present in the precipitates with average size of 2.3 by 1.4 μm. The cluster size averaged 1.8 by 1.4 μm with 1 nM sGP. For the NC sample, only 80 nm AuNP monomers but no clusters were observed in the precipitates. The aggregate sizes had a large distribution, probably due to a random aggregation process and sample preparation, and thus was not ideally correlated with the sGP concentration.

Drop-Casting on Glass Slides for Optical Inspection.

The AuNP assay upper-level liquid (1 μL, sGP mono-binder, 80 nm AuNP, n_{AuNP}=0.036 nM) with sGP from 1 pM to 1 μM in PBS were drop-casted on a 1 mm thick glass slide for colorimetric and spectrometric inspections. It can be observed that the dried sample spots displayed light red color, and their transparency increased from around 10 nM (spot 3) and became readily differentiable to the naked eye at 1 μM (spot 1) compared to the reference NC sample (spot 8). We then measured the extinction spectra of each drop-cast spots and extracted the extinction peak intensity at LSPR resonance. The extinction spectra featured AuNP LSPR peaks, similar to those upper-level liquid measurements in the PDMS well plate, but the peak intensity was about one order of magnitude smaller attributed to a significantly shorter optical path (estimated ˜300 μm) compared to the PDMS well plate (˜3 mm).

These samples were stored at room temperature (25° C.) over 12 weeks and re-inspected, and the optical signals were found in general to be consistent over such an extended period with only slight change. The slight increase in extinction, observed especially at the lower sGP concentration, was possibly due to shrinking of the drop cast spot from dehydration as the sample was exposed to dry air. Nevertheless, this also showed the feasibility of quantitative detection of sGP down to 350 pM LoD with a broad dynamic range in detection (100 pM to 1 μM) using a simple and small solid-state sample carrier.

In comparison, we also performed drop-casting of 60 nm AuNPs on glass slides, which also shows performance similar to the detection in the PDMS well plate (FIG. 9).

Drop-Casting on Gold Film for SEM and Dark-Field Inspection

Scanning electron microscopy (SEM) was employed to investigate the AuNP aggregation in the upper-level liquid. Here 1 μL of 80 nm AuNP assay colloids (n_{AuNP}=0.036 nM, 1×PBS) with targeted sGP (1 pM to 1 μM) were drop-cast onto an oxygen plasma treated gold surface and subsequently dried in air. Gold surface was selected due to its high electron conductivity that dramatically improves the contrast and resolution in imaging. Only AuNP monomers but no large aggregates were observed from the SEM images, confirming that the majority, if not all, of the aggregates should precipitate at the bottom of the tubes. Further, the 80 nm AuNPs were recognized and counted through image analysis, and their density was statistically determined from ten SEM images (total area 10×8.446×5.913 μm²) at each sGP concentration. Clearly, the AuNP density decreased at a higher sGP concentration, i.e. from 1.97 μm⁻² at about 10 pM to 0.26 μm⁻² at 100 nM and finally saturated to 0.24 μm⁻² at about 1 μM or above, which was in accordance with extinction spectrometric measurements of both upper-level liquid samples and glass slide drop-cast samples. The limit of detection derived from SEM characterization was estimated to be about 150 pM, comparable but slightly higher than the upper-level liquid extinction characterization for the same 80 nm AuNPs in PBS, possibly due to increased variance in nanoscale level characterization and limited sampling data.

Further, the drop-cast samples on gold surface were also analyzed by dark field scattering imaging. Here LSPR mediated scattering of incident light from AuNPs on gold surface can directly indicate the density of AuNP, based on the density of bright spots on dark field imaging. The dark field scattering images were undergoing image process to improve the contrast for spots, which were then counted using MATLAB code and averaged over 10 images (captured area 62.5 μm×62.5 μm in each image). It was observed that the density of bright spots dropped as sGP concentration increased, consistent with SEM observation. The dark field imaging method was estimated to be capable of detecting sGP with a sensitivity about 1 nM.

Drop-Casting on Gold Film in Detecting sGP in 1×PBS with 100 nm AuNPs.

To investigate the feasibility of different AuNP sizes on sensing, we prepared 100 nm AuNP assay samples, drop-cast them on 1 mm glass slides and gold films, and characterized by UV-visible spectrometer, SEM, and dark field scattering imaging. The 100 nm AuNP concentration in assay colloid was 0.019 nM. The characterizations followed protocols described in main context method: UV-visible spectra, SEM imaging, and dark field scattering imaging characterizations. The measurement results in general showed comparable sensitivity (sub 1 nM) to that of 80 nm AuNPs and consistent with detection in PDMS well plate.

Back-of-the-Envelope Estimation to Understand the Detection Limits

Our experimentally determined upper and lower limits in detection are thought to be correlated to the multivalent binding nature of the detection process. Suppliers informed that up to 120, 270, 460, and 730 streptavidin are bound on 40, 60, 80 and 100 nm diameter NPs, although the effective numbers are expected to be smaller. This large number of binding sites on the AuNPs makes them a strong multivalent binding sensor that is highly favorable to AuNP aggregation, even at low antigen concentration.

With very small amounts of antigens, the ultimate lower limit of detection occurs when NP precipitation decreases the AuNP monomers in the suspension significantly enough to produce a signal that is distinguishable from noise or fluctuation. This value is expected to be dependent on both the dynamic antigen-antibody binding process and the experimental setup. For example, assuming a very high affinity in sGP binding (ignoring dissociation) and four sGP bound to each aggregating AuNP (about 3-5 at 1 nM from TEM) and setting 3% optical extinction change as the detection threshold to overcome signal variation, we could roughly estimate the LoD as 275×(3%/45%)×4=70 pM or 36×(3%/45%)×4=10 pM for 40 and 80 nm AuNPs, which are comparable to our experimental analysis. Similarly, the upper limit of detection could be estimated when the AuNPs are completely saturated with the analyte, i.e. 120×0.275=33 nM and 460×0.036=16 nM for 40 and 80 nm AuNPs, also comparable to but smaller than experimental values. The above back-of-the-envelope analysis is helpful to provide intuitive understanding of the measured dynamic range and detection limits, but it is also quite limited because it ignores the dynamic association and dissociation processes which are thought to be dependent on both the NP size and antigen-nanobody binding characteristics.

Modeling to Understand Sensing Physics

Rate Limiting Reaction Steps and Diffusion.

Using 80 nm AuNP as an example, we first attempted to identify the key rate determining step in the sensing mechanism. Each of the 80 nm AuNPs has ˜460 nanobodies on their surface and behaves as a multivalent sGP-binding pseudo-particle that diffuses and conjugates to each other via sGP-mediated bridging. This triggers formation of AuNP dimers oligomers, and eventually large clusters, which precipitate at the bottom of microcentrifuge tube as gravity gradually overtakes fluidic drag force. Therefore, the reaction determining steps during the sensing process include the antigen diffusion, AuNP diffusion, antigen-AuNP binding, AuNP clustering, and AuNP precipitation.

The diffusivities of AuNPs and antigens can be estimated from the Stokes-Einstein equation D=kT/(3πηd), where kT is the thermal energy at room temperature (˜4.1×10⁻²¹ Joule at 300K), η is the solution viscosity (˜1.7×10⁻³ N·sec/m² assuming 20% glycerol in water to estimate the buffer effect), and d is the particle diameter². The diffusivity is estimated Dₐ˜5.12×10⁻¹¹ m²/s for a 5 nm protein and D_{NP} ˜3.2×10⁻¹² m²/s for an 80 nm AuNP. We can further estimate the diffusion length Lₐ, i.e. the distance for analyte to collide with AuNPs, as the smaller of the inter-protein separation Lₚ and inter-AuNP separation L_{NP}. L_{NP} was calculated in the range of 2 to 5 μm for 40 to 100 nm AuNPs used in our experiment following

$${L}_{NP} = \sqrt[3]{\frac{6 V}{π}} = \sqrt[3]{\frac{6}{π} \frac{1}{{c}_{NP} {N}_{A}}} ,$$

where V is volume estimated for each NP (assuming a sphere), c is the NP molar concentration, and N_{A} is the Avogadro number. Clearly, L_{NP} is determined by the NP concentration and thus is a constant once the assay is designed. Similarly, Lₚ depends on analyte concentration and can be calculated as ˜2 μm at a low sGP concentration (<100 pM) but <100 nm at a higher concentration (>1 μM). Therefore, Lₐ is mainly determined by the protein concentration, and the diffusion time tₐ˜Lₐ²/Dₐ is found to be only 0.1 to 0.2 sec, much shorter than the experimentally determined incubation assay time (3 to 7 hours).

It is important to compare here with conventional surface-incubation based assays, such as ELISA and SPR, where according to the non-slip boundary condition the surface velocity is close to zero. Differently, in our case the AuNP continues to diffuse, and its thermal velocity can be estimated by υ_{NP}=\sqrt{KT/m_{NP}}. Because of its small mass (m_{NP}=5.18×10⁻¹⁵ g for an 80 nm AuNP), there is a significantly large thermal velocity for the AuNPs υ_{NP}˜0.028 m/sec. Given this velocity and the small Lₐ, we can understand that in fact the AuNPs should be constantly colliding with antigens and other nanoparticles, promoting effective mixing and antigen binding. Therefore, the diffusion process will not be a rate limiting step here, although they could significantly limit the assay time of ELISA and SPR. In another word, the antigen binding process behaves totally differently from that on an infinitely large surface in ELISA and SPR, and the measured kₒₙ, k_{off} from ELISA is limited to the surface bound molecular interactions and cannot fully predict what happens at the nanometer scale in our case. Such phenomena have been observed that the binding kinetics in solution could be significantly different from that on surface, which is attributed to mass transport and other factors. On the other hand, given the high binding affinity of analyte-ligand complex in this proposed work, we can reasonably hypothesize that the association process of this complex is fast (e.g., G protein binds to GPCR receptors within ˜0.3 sec), thus also unlikely the limiting step.

Simplified Mathematical Modeling of AuNP Aggregation.

Here we adapted Smoluchowski's coagulation equation and modified the equation to describe our reversible AuNP aggregation process, using sGP sensing as an example. The modified equation is:

$$\frac{{dn}_{i}}{dt} = \frac{1}{2} {\sum }_{j = 1}^{i - 1} {k}_{i - j , j} {n}_{i - j} {n}_{j} - {\sum }_{j = 1}^{∞} {k}_{i , j} {n}_{i} {n}_{j} + {k}_{off} {n}_{i + 1} - {k}_{off} {n}_{i} \qquad ( 1 )$$

Where nᵢ(t) is the concentration of aggregates consisting of i AuNPs, k_{i,j} is the coagulation kernel for the aggregation of clusters consisting of i AuNPs and j AuNPs, k_{off} is the dissociation constant of sGP49-sGP conjugation (k_{off}=1.88×10⁻⁴ s⁻¹) derived from ELISA measurement. According to Brownian diffusion theory, the coagulation kernel k_{i,j} is described as:

$${k}_{i , j} = \frac{2}{3} P \frac{{k}_{B} T}{η} {( ij )}^{γ} ( {m}_{i}^{\frac{1}{{d}_{f}}} + {m}_{j}^{\frac{1}{{d}_{f}}} ) ( {m}_{i}^{- \frac{1}{{d}_{f}}} + {m}_{j}^{- \frac{1}{{d}_{f}}} ) \qquad ( 2 )$$

Where P is the probability of aggregation per collision, k_{B} is Boltzmann constant, T is temperature, η is dynamic viscosity of colloid buffer (˜1.7×10⁻³ Ns/m² for 20% glycerol in water), i and j are numbers of AuNP in each cluster, mᵢ and mⱼ are mass of each clusters, d_{f} is the fractal dimension (˜2.1 for a typical densely aggregated cluster). Here P could be estimated as 0.0165 if using the ELISA-determined kinetic parameter kₒₙ=4.07×10⁴ M⁻¹s⁻¹. This value is found a serious underestimate because the mass transport and antigen binding process in in-solution Nano2RED assay are significantly more effective than that for ELISA, as discussed in further herein. Indeed, we found using such a small P value could not accurately predict the assay time observed in Nano2RED. Instead we used P=1, meaning every collision of sGP and sGP49-functionalized AuNPs will result in antigen binding. Such an assumption led to good agreement between theory and experimental observations. In fact, such a high binding efficiency is reasonable given the multivalence nature of AuNP sensors. The multivalence effectively creates a much higher “functional affinity” compared to the intrinsic affinity by monovalent binding, and could yield virtually irreversible binding process.

In equation 1, the first two terms in the right side directly come from Smoluchowski's equation that describe the AuNP aggregation process. The other two terms on the right side are added terms to describe the reversible dissociation of AuNP aggregates. For simplification, we considered only the dissociation of a cluster with N AuNPs to form a cluster of N−1 AuNPs and a monomer released back to colloid (i.e. N→N−1,1). Although the breakdown of clusters to clusters with other arbitrary numbers of AuNPs is possible (N→N−i, i), such breakdown requires multivalent sGP-sGP49 dissociation, hence the effective dissociation rate is likely to be much smaller. Moreover, a further simplification of the model by considering only the low-order oligomers and monomers interactions is justified by the fact that the concentration of nᵢ with higher i numbers (higher order) is small due to precipitation. Therefore, we could calculate the AuNP monomer concentration based on the simplified equation set and thus estimate the extinction signals by considering only the low-order oligomer-monomer interactions. This assumption is especially valid at the beginning when the assay is mixed with sGP protein, where the concentration of higher order oligomers and large clusters is near 0. Our simplified model incorporates an evolution of large cluster formation, and the equation sets are shown below:

$$\begin{matrix}\frac{{dn}_{1}}{dC} = - 2 {k}_{11} {n}_{1}^{2} - {\sum }_{i = 2}^{∞} {k}_{1 i} {n}_{i} {n}_{1} + 2 {k}_{off} {n}_{2} + {\sum }_{i = 3}^{∞} {k}_{off} {n}_{i} & ( 3 ) \\ \frac{{dn}_{2}}{dC} = 2 {k}_{11} {n}_{1}^{2} - {k}_{12} {n}_{1} {n}_{2} + {k}_{off} {n}_{3} - 2 {k}_{off} {n}_{2} & ( 4 ) \\ \frac{{dn}_{3}}{dC} = {k}_{12} {n}_{1} {n}_{2} - {k}_{13} {n}_{1} {n}_{3} + {k}_{off} {n}_{4} - {k}_{off} {n}_{3} \\ … & ( 5 ) \\ \frac{{dn}_{i}}{dC} = {k}_{1 i - 1} {n}_{1} {n}_{i - 1} - {k}_{1 i} {n}_{1} {n}_{i} + {k}_{off} {n}_{i + 1} - {k}_{off} {n}_{i} & ( 6 )\end{matrix}$$

By solving the equations above, we obtained the time-dependent monomer concentration, assuming 0.036 nM AuNP colloid in detecting 10 nM sGP, and further converted the concentration to optical extinction. Intuitively, the solver of this equation set showed that the monomer concentration versus time is quasi-exponential. The aggregation time constant T_{agg}, defined as time required for concentration of AuNP monomer drop to

$${c}_{equillibrium} + \frac{1}{e} ( {c}_{0} - {c}_{equillibrium} ) ,$$

is 0.87 hour.

Further, we calculated the monomer concentration versus time for 1.8 nM 80 nm AuNP assay in detecting 10 nM sGP. In this case, T_{agg} is significantly shortened to 0.024 hour, or ˜36 times smaller compared to T_{agg} using 0.036 nM AuNPs.

Sedimentation Time.

As AuNPs cluster, the gravitational force overcomes fluidic drag, and large clusters precipitate to form sedimentation and continuously deplete AuNPs and sGP proteins in the colloid until reaching equilibrium. The sedimentation time of this progression can be estimated from the solution of the Mason-Weaver equation by τ_{sed}=z/(s·g) where z is the precipitation path (the height of colloid liquid), g is the gravitation constant, s is the sedimentation coefficient

$$s = \frac{{d}^{2}}{18 η} ( {ρ}_{M} - {ρ}_{w} )$$

(d is the aggregate diameter, η is the dynamic viscosity of the colloid buffer, ρₘ and ρ_{w} are the density of aggregate and colloid buffer, respectively). Clearly, the large density contrast between gold (19.3 g/cm³) and buffer (˜1 g/cm³) is also beneficial to improve the sedimentation coefficient s. Given z ˜3.5 mm for 16 μL liquid in a microcentrifuge tube, we calculated that τ_{sed} decreases from 26.0 hours for an 80 nm AuNP monomer to 1.0 and 0.3 hours for 400 nm and 800 nm diameter clusters, respectively.

Rapid Detection

Impact of Incubation Time.

The optical images of the microcentrifuge tubes indicated the color contrast was high enough to be immediately resolved by the naked eye after vortex mixing. We have analyzed the peak extinction of the assay upper-level liquid at different incubation times, and the extinction was found to be 0.145 right after vortex, completely distinguishable from the NC sample (0.536), and it gradually decreased to 0.110 as incubation time was extended to 20 min. The analysis show that further incubation could be used as an option to moderately improve the signal contrast, but could possibly be skipped when testing speed is extremely important.

In addition, the rapid sGP detection scheme was found to be reproducible in fetal bovine serum (5% FBS), and it produced similar results in 1×PBS buffer. In both cases, sGP >1 nM can be accurately read out by the naked eye either in tubes or in a PDMS well plate.

Portable UV-Visible Spectrometer as Readout.

The portable UV-visible spectrometer system (FIG. S13) consists of a smartphone sized Ocean Optics UV-visible spectrometer (8.8×6.3×3.1 cm³), a lamp source module (15.8×13.5×13.5 cm³), alignment clamps, and an electronic recording device (such as a laptop or a smart phone). Here, 80 nm AuNP colloid (30 μL) is mixed with sGP testing buffer (10 μL 5% FBS), vortexed and centrifuged at 1,200×g (3,500 rpm) for 1 min. After 20 minutes of incubation, the assay colloids were vortexed for 15 seconds and the upper-level liquid were loaded into 4 mm-diameter wells on a 3 mm thick PDMS plate. The light from the lamp transmitted through the colloid, whereas the rest area of the diced fused silica was covered in black to block stray light transmission. Transmitted light was collected by the spectrometer through a fiber waveguide. The extinction spectra, measured by portable spectrometer (Ocean Optics), were in general highly consistent with that measured by microscope-coupled spectrometer (Horiba iHR320), with a slightly increased signal noise. The extinction peak values at the resonance wavelength (˜559 nm) of the two measurements were in high agreement, with a small difference within 15.8%. This could possibly be attributed to different signal collection setup (10× objective with NA of 0.3 in lab-based spectrometer versus waveguide collecting a nearly collimated beam in portable spectrometer). The optical signals measured by portable spectrometer were able to distinguish sGP at 100 pM (E_{100 pM}=0.509) from the reference (E_{NC}=0.542, no sGP), with a dynamic range (10 pM to 1 μM) and limit of detection (˜42 pM) comparable to that of the rapid detection of sGP in serum and 1×PBS.

Rapid sGP Detection with a Portable, Electronic Readout Device

Extinction spectrometric analysis provides quantitative and accurate diagnostics but requires bulky spectrometer systems that are more suitable for lab use. We demonstrated the feasibility of detecting sGP in FBS using a cost-efficient, portable UV-visible spectrometer system for field deployment. Additionally, we developed a homemade LED-photodiode based electronic readout system with significantly reduced system cost to deliver accurate and sensitive detection results comparable to a lab-based spectrometer system (FIGS. 11I-K). Here, an LED light source emitted narrowband light at the AuNP extinction peak (λ_{P}=560 nm, FWHM_{P}=40 nm), which transmitted through the upper-level assay liquid and then was collected by a photodiode (FIG. 11I). As a result, a photocurrent or photovoltage was generated on a serially connected load resistor in a simple circuitry that can be easily integrated and scalably produced. In practice, we 3D-printed a black holder to snug-fit a microcentrifuge tube, and mounted the LED and photodetector on two sides of the holder (FIG. 11J). The LED and photodiode were powered by alkaline batteries (3V and 4.5V, respectively), and the bias voltages were set to ensure wide-range detection of sGP proteins without saturating the photodetectors. Using 80 nm sGP49-functionalized AuNP Nano2RED assays, sGP was detected in diluted FBS (5%) by reading the photovoltage signals with a handheld multimeter (FIG. 12K, V_{R}, in black). Compared to lab-based spectrometric readout (in blue), the electronic readout displayed identical dynamic range, but slightly improved LOD (27 pM compared to 80 pM).

Nano2RED with Co-Binders and Testing in Different Biological Buffers

The Impact of Biological Buffer Concentration.

Using FBS as a buffer and sGP49 functionalized 80 nm AuNPs for sensors, we have investigated the effect of buffer concentration. We found that high concentration FBS (50%) produced smaller optical contrast for readout compared to low concentration, <20% FBS. Further, 5% FBS displayed more consistent results and a slightly larger dynamic range for detection. For consistency in comparison, we chose 5% as the concentration of all biological buffers to be tested in the co-binder experiments.

sGP Detection Using Co-Binder sGP49/sGP7.

Two different biotinylated nanobodies, sGP49 and sGP7, were surface functionalized to streptavidin coated AuNP similar to the method described earlier for creating two different sets of functionalized AuNP colloidal solutions. The concentration of functionalized AuNP colloids were re-adjusted to get an optimal extinction level. sGP stock solution underwent serial dilution to create an sGP analyte solution with concentrations of 4 uM to 400 fM in selected detection media. The final composition of PBS detection media was composed of 1×PBS, 20% v/v glycerol and 1 wt % BSA while that of FBS, HPS (Human pooled serum), and WB (Whole blood) detection media had a final concentration of 1×PBS, 20% v/v glycerol and 1 wt % BSA and 20% of either FBS, HPS, or WB which resulted in a final concentration of FBS, HPS, or WB in the detection assay to be 5%. Solutions of sGP49-functionalized AuNPs, sGP7-functionalized AuNP, and sGP were mixed in a 500 uL Eppendorf tube at a ratio of 3:3:2 and thoroughly vortexed. After mixing, the detection assay was centrifuged (accuSpin Micro 17, Thermo Fisher) at 3,500 rpm (1,200×g) for 1 minute. AuNPs were highly concentrated at the bottom of Eppendorf tube. After 20 minutes of incubation, the colloidal assay was vortexed (mini vortexer, Thermo Fisher) at 800 rpm for 15 seconds to thoroughly remix free AuNP monomers into the colloid. Following this, spectrometric and electronic characterizations were done in a way similar to that described previously.

Detection Error and LOD in Phage ELISA.

To determine the Phage ELISA LOD analysis similar to the method described in previous section was performed (Table 2). For consistency in LOD analysis, we analyzed the results with three different methods for sigma estimation. The first method is the traditional way of determining LOD, which only takes into account the standard deviation of blank (or NC) sample measurement (termed σ_{NC}). The two additional methods, i.e., Pooled Sigma All (σ_{PSA}) and Pooled Sigma 4 (σ_{PS4}) employ a pooled variance method which is used for statistical analysis of different populations but with similar variances to give a more robust and consistent result. While σ_{PSA} takes into account all of the samples, σ_{PS4} takes onto account only the last four samples.

Here for ELISA, the measurement noise is strongly correlated to signal level and sample concentration (Table 2). As a result, σ_{PSA} is quite high due to the consideration of high-concentration samples. Yet the use of σ_{NC} makes the result very susceptible to experimental errors in measuring the NC sample, which could seriously affect the LOD by orders of magnitude given a small signal difference at low sample concentration. Comparatively, σ_{PS4} provides a more balanced estimation for reagent characterization, because the last 4 sample data (including the NC sample) have similar signal and noise levels and the average in fact provides a more consistent estimation of experimental errors.

Detection Error and LOD in Rapid Test.

For consistent data acquisition, it was imperative that the spectrometric data acquisition be done as soon as possible in order to minimize error introduced by gravitational precipitation of AuNP. Here it was noticed that the traditional method of determining LOD, i.e. σ_{NC}, negatively affected the consistency in LOD determination (Table 3). This can be attributed to the nature of data collection in that optical focusing varies from one measurement to the next. In comparison, σ_{PSA} and σ_{PS4} provide better consistency and robustness in determining the standard deviation required for LOD measurement. This is particularly true when the error measured from the NC sample is too high or too low compared to the average errors measured across the whole sample set. Here, we choose the σ_{PSA} method for further analysis of the data because of its better data consistency. Table 2 shows the summary of the analysis while FIG. 40 shows the LOD determination using the different calculated SD values.

TABLE 3
LOD analysis of Ebola sGP detection by Nano2RED

| | | Readout | σ (A.U.) | | | LOD (pM) | | |
|---|---|---|---|---|---|---|---|---|
| Antigen | Media | method | σ_{PS4} | σ_{PSA} | σ_{NC} | σ_{PS4} | σ_{PSA} | σ_{NC} |
| Ebola | PBS | Spectrometric | 0.005211 | 0.005747 | 0.0042 | 0.133 | 0.161 | 0.0913 |
| sGP | FBS | | 0.007094 | 0.005944 | 0.00914 | 1.65 | 1.05 | 3.75 |
| | HPS | | 0.004836 | 0.00548 | 0.00558 | 1.09 | 1.26 | 1.29 |
| | HPS | Electronic | 0.004459 | 0.004518 | 0.00483 | 0.134 | 0.135 | 0.142 |
| | WB | Spectrometric | 0.005841 | 0.005276 | 0.00701 | 18.24 | 16.9 | 21.44 |

Detection of sGP in Serum and Blood

We further evaluated the use of co-binding nanobodies in sGP sensing (FIG. 12), i.e., sGP49/sGP7 for sGP (FIG. 9), and performed Nano2RED tests in different buffers, including PBS, FBS, human pooled serum (HPS), and whole blood (WB). The incubation and rapid assay formats with the assay performance, instrument costs, and LODs were summarized in Table 1, and additional data on measurement variance (sigma) and LOD were summarized in Tables 2 and 3. There are several notable observations. First, using sGP sensing as an example and comparing to previously reported results (Table 3), Nano2RED with spectrometric and electronic readout consistently produced ˜130 fM to 1.3 pM LOD (or ˜10 to ˜100 pg/mL) in PBS, FBS, and HPS. It is noted that a very recently reported co-binder-based D4 assay format reported ˜30 pg/mL LOD in human serum, and was able to detect the Ebola virus earlier than PCR in a monkey model. In LOD comparison, the sensitivity of Nano2RED (˜10 pg/mL with electronic readout) is even better, indicating its competitiveness in high-precision diagnostics.

Additionally, our study (Table 1) also revealed the importance of a systematic assay design strategy, from molecular binding to signal transduction and readout, to optimize antigen detection. It is clear that the co-binder pair improved the LOD by 10 to 100 times compared to the mono-binder (sGP49, k_{D} 4.6 nM), despite a relatively low k_{D} of 199 nM for the second binder (sGP7) (FIG. 9). This improved sensitivity is likely because the co-binders have a favorable, non-competitive binding configuration that serves to improve antigen binding and AuNP aggregation. Uniquely, the use of a portable and inexpensive electronic readout did not negatively affect the LOD of Nano2RED, but rather improved it compared to spectrometric readout (FIG. 12I, and Table 1). This can be attributed to smaller 3-sigma errors in the electronic readout (Table 3), partly due to a larger signal fluctuation in optical imaging caused by manual operation, such as in focusing. Here, the electronic signal is mainly dependent on the circuit elements but much less dependent on operators' judgement, and thus potentially more reliable and accurate. In addition, the use of biological buffers could also affect detection. For Ebola sGP, the LOD increased by about 5-10 times in serum (FBS and HPS) than in PBS, and further increased by another 10 times in WB. Additionally, the colorimetric readout by the naked eye was capable of detecting both antigens in serum at concentrations higher than 100 pM or 1 nM (FIG. 12); however, it became challenging to do so in WB, mainly due to the fact that WB absorbs in short wavelengths and causes background color interference with AuNPs (FIG. 12J-K). However, the spectrometric readout could still readily identify the sGP extinction signals from the background WB absorption for accurate detection (FIG. 12I for sGP), indicating the feasibility of Nano2RED for field use with minimized sample preparation.

Fundamentally different from conventional high-sensitivity antigen diagnostics that usually require bulky and expensive readout systems, as well as long assay time, Nano2RED is an affordable and accessible diagnostic technology. For example, ultrasensitive sGP sensing using NP-enhanced fluorescent readout would require 3-4 hours of image processing to reduce noise for optimal sensing, and these fluorescent systems usually require cubic meter space and cost $40,000 or more (a high-end fluorescent camera with high signal-to-noise ratio is ˜$25,000). Similarly, a D4 co-binder assay requires a lab-based bulky fluorescent system and ˜60 min assay time to achieve PCR-comparable diagnostic sensitivity. Its sensing performance drops ˜10 times to 100 pg/mL when using a customized fluorescent system, which costs ˜$1,000 and occupies ˜3,000 cm³. The performance further decreases to 6,000 pg/mL when using LFA with colorimetric readout. Clearly standing apart from the rest, Nano2RED utilizes miniaturized and low-cost semiconductor devices for signal readout rather than a fluorescent system. Therefore, it has a very small footprint (4 cm³ for tube holder, or <100 cm³ for the whole system, including batteries and meters, which all could be miniaturized on a compact circuit board in the future), is very low cost (LED and photodiode each <$1 here, but can be <$0.1 when used at large scale, with the total system cost estimated well below $5), and offers a rapid readout (5 to 20 min, depending on incubation time after centrifugation). Further, the electronic readout is more accurate than the colorimetric readout, more accessible, without color vision limitations, and more readily available for data storage in computers or online databases for real-time or retrospective data analysis. Additionally, we have estimated the reagent cost in Nano2RED is only about $0.01 per test (Supplementary section 9), since it requires only a small volume (˜20 μL) of reagents.

We tested sGP against GP1,2, a homotrimer glycoprotein transcribed from the same GP gene and sharing its first 295 residues with sGP¹⁶, both in FBS (FIGS. 12 D-F and M-O). The majority of GP1,2 can be found on virus membranes whereas a small portion is released into the patient's bloodstream. The close relevance of GP1,2 to sGP makes it a very strong control molecule to assess our assay's specificity. Indeed, GP1,2 did not produce detectable signals unless higher than 1 nM, indicating a high selectivity over a broad concentration range (100 fM to 1 nM, or 4 logs) where minimal nanobody binding or AuNP aggregation occurred. A high assay specificity is crucial for minimizing false positive diagnosis of infectious diseases, which could lead to unnecessary hospitalizations and even infections. Considering that 10 nM and higher sGP concentration is typical for EVD patients, Nano2RED is particularly suitable for high-speed mass screening of EVD susceptible populations.

Conclusions and Outlook

We have demonstrated a generalizable and rapid assay design and pipeline that combines fast affinity reagent selection and production with nanometer-scale theoretical analysis and experimental characterization for optimized sensing performance. Synthetic, high-affinity, co-binding nanobodies, which could be quickly produced by a phage display selection method from a premade combinatorial library for any given antigen, proved to be effective in detecting dimeric Ebola sGP proteins. The Nano2RED utilized unique signal transduction pathways to convert biological binding into electronic readout. Using simple electronic circuitry, it starts with AuNP aggregation (governed by dynamic antigen-nanobody interactions described by Langmuir isotherm), which triggers AuNP cluster sedimentation (explained by Mason-Weaver equation), and then enables AuNP-concentration-dependent optical extinction (following Beer-Lambert law). The use of AuNPs for in-solution testing serves to greatly facilitate fluidic transport and antigen binding at nanometer scale. Nano2RED eliminates the need for long-time incubation due to slow analyte diffusion in conventional ELISA and other plane surface-based assays, as well as its associated cumbersome washing steps. The introduction of brief centrifugation and vortex mixing further greatly shortens the aggregation and sedimentation time, enabling rapid tests (within 5 to 20 min) without sacrificing sensitivity or specificity. Our data showed that Nano2RED is highly sensitive (sub-picomolar or ˜10 pg/mL level for sGP) and specific in biological buffers while also affordable and accessible. Importantly, the portable electronic readout, despite being very simple and inexpensive (<$5), proved to be more reliable and sensitive than colorimetric and even spectrometric readout. Nano2RED can be applied for lab tests to detect early-stage virus infection at a high sensitivity potentially comparable to PCR. It can also be used for high-frequency at-home or in-clinic diagnostics, as well as in resource-limited regions, which could greatly enhance control of disease transmission. The digital data format will also reduce human intervention in data compiling and reporting, while facilitating fast and accessible data analysis.

Methods

Materials. Phosphate-buffered saline (PBS) was purchased from Fisher Scientific. Bovine serum albumin (BSA) and molecular biology grade glycerol were purchased from Sigma-Aldrich. Fetal bovine serum (FBS) was purchased from Gibco, Fisher Scientific. FBS was used without heat inactivation to best reflect the state of serum collected in field. Polyvinyl alcohol (PVA, M_{W} 9,000-10,000) was purchased from Sigma-Aldrich. Sylgard 184 silicone elastomer kit was purchased from Dow Chemical. DNase/RNase-free distilled water used in experiments was purchased from Fisher Scientific. Phosphate Buffered Saline with Tween 20 (PBST), Nunc MaxiSorp 96 well ELISA plate, streptavidin, 1% casein, 1-Step Ultra TMB ELISA substrate solution, and isopropyl-β-D-galactopyranoside (IPTG) were purchased from Thermo Fisher Scientific. HRP-M13 major coat protein antibody was purchased from Santa Cruz Biotechnology. Sucrose and imidazole were purchased from Sigma-Aldrich. A 5 mL HisTrap column, HiLoad 16/600 Superdex 200 pg column, and HiPrep 26/10 desalting column were purchased from GE Healthcare. BirA-500 kit was purchased from Avidity. Streptavidin (SA) Biosensors were purchased from ForteBio. The streptavidin functionalized AuNPs, dispersed in 20% v/v glycerol and 1 wt % BSA buffer, were purchased from Cytodignostics. Thiolated carboxyl polyethylene glycol linker was self-assembled on AuNP through a thiol-sulfide reaction. Streptavidin was then surface functioned through amine-carboxyl coupling by N-Hydroxysuccinimide/1-Ethyl-3-(3-dimethylaminopropyl) carbodiimide (NHS/EDC) chemistry.

Phage display selection. sGP (GenScript) protein binder selection was done according to previously established protocols. In brief, screening was performed using biotin and biotinylated target protein-bound streptavidin magnetic beads for negative and positive selections, respectively. Prior to each round, the phage-displayed nanobody library was incubated with the biotin-bound beads for 1 h at room temperature to remove off-target binders. Subsequently, the supernatant was collected and incubated with biotinylated-target protein-bound beads for 1 h. Beads were washed with 10×0.05% PBST (1×PBS with 0.05% v/v Tween 20) and phage particles were eluted with 100 mM triethylamine. A total of three rounds of biopanning were performed with decreasing amounts of antigen (200 nM, 100 nM, 20 nM). Single colonies were picked and validated by phage ELISA followed by DNA sequencing.

Single phage ELISA. ELISAs were performed according to standard protocols. Briefly, 96 well ELISA plates (Nunc MaxiSorp, Thermo Fisher Scientific) were coated with 100 μL 5 μg/mL streptavidin in coating buffer (100 mM carbonate buffer, pH 8.6) at 4° C. overnight. After washing with 3×0.05% PBST (1×PBS with 0.05% v/v Tween 20), each well was added to 100 μL 200 nM biotinlyated target protein and incubated at room temperature for 1 h. Each well was washed by 5×0.05% PBST, blocked by 1% casein in 1×PBS, and added to 100 μL single phage supernatants. After 1 h, wells were washed by 10×0.05% PBST, added to 100 μL HRP-M13 major coat protein antibody (RL-ph1, Santa Cruz Biotechnology; 1:10,000 dilution with 1×PBS with 1% casein), and incubated at room temperature for 1 h. A colorimetric detection was performed using a 1-Step Ultra TMB ELISA substrate solution (Thermo Fisher Scientific) and OD450 was measured with a SpectraMax Plus 384 microplate reader (Molecular Devices).

Mono-binders purification and biotinlyation. sGP7 and sGP49 mono-binders were expressed as a C-terminal Avi-tagged and His-tagged form in E. coli and purified by Ni-affinity and size-exclusion chromatography. In brief, E. coli strain WK6 was transformed and grown in TB medium at 37° C. to an OD600 of ˜0.7, then induced with 1 mM isopropyl-β-D-galactopyranoside (IPTG) at 28° C. for overnight. Cell pellets were resuspended in 15 mL ice-cold TES buffer (0.2 M Tris-HCl pH 8.0, 0.5 mM EDTA, 0.5 M sucrose) and incubated with gently shaking on ice for 1 h, then added to 30 mL of TES/4 buffer (1:4 dilution of the TES buffer in ddH₂O) and gently shaken on ice for 45 min. Cell debris was removed by centrifugation at 15,000×g, 4° C. for 30 mins. The supernatant was loaded onto a 5 mL HisTrap column (GE Healthcare) pre-equilibrated with the lysis buffer (50 mM sodium phosphate, pH 8.0, 300 mM NaCl, 10 mM imidazole, 10% glycerol). The column was washed with a washing buffer (50 mM sodium phosphate, pH 8.0, 300 mM NaCl, 20 mM imidazole, 10% glycerol) and then His-tagged proteins were eluted with an elution buffer (50 mM sodium phosphate, pH 8.0, 300 mM NaCl, 250 mM imidazole, 10% glycerol). Eluates were loaded onto a HiLoad 16/600 Superdex 200 pg column (GE Healthcare) pre-equilibrated with a storage buffer (1×PBS, 5% glycerol). Eluted proteins were concentrated, examined by SDS-PAGE, and quantified by a Bradford assay (BioRad), then flash frozen in 100 μL aliquots by liquid N₂ and stored at −80° C.

The purified protein was biotinylated by BirA using a BirA-500 kit (Avidity). Typically, 100 μL BiomixA, 100 μL BiomixB, and 4 μL 1 mg/mL BirA were added to 500 μL protein (˜1 mg/mL), and adjusted to a final volume of 1000 μL with nuclease-free water (Ambion). The biotinylation mixture was incubated at room temperature for 1 h and then loaded onto a HiPrep 26/10 desalting column (GE Healthcare) pre-equilibrated with a storage buffer (1×PBS, 5% glycerol) to remove the free biotin.

Binding kinetics analysis. The four mono-binders' binding kinetics were analyzed using an Octet RED96 system (ForteBio) and Streptavidin (SA) biosensors. 200 nM biotinylated sGP target protein was immobilized on SA biosensors with a binding assay buffer (1×PBS, pH 7.4, 0.05% Tween 20, 0.2% BSA). Serial dilutions of mono-binder were used for the binding assay. Dissociation constants (K_{D}) and kinetic parameters (kₒₙ and k_{off}) were calculated based on global fit using Octet data analysis software 9.0. For co-binder validation, sGP bound SA biosensors were first dipped into the sGP49 wells 750 s for saturation, then incubated with sGP7 for 750 s.

Determination of detection sensitivity using a co-binder sandwich ELISA assay. In order to validate and determine the detection sensitivity of co-binders, a sandwich ELISA-like assay was performed. Briefly, 96 well ELISA plates (Nunc MaxiSorp, Thermo Fisher Scientific) were coated with 100 μL 5 μg/mL streptavidin in coating buffer (100 mM carbonate buffer, pH 8.6) at 4° C. overnight. After washing with 3×0.05% PBST (1×PBS with 0.05% v/v Tween 20), each well was added to 100 μL 200 nM biotinlyated sGP49 (˜100 nM) protein and incubated at room temperature for 1 h, then washed with 5×0.05% PBST and added to 100 μL serial dilutions (0 to 500 nM) of sGP protein and incubated at room temperature for 1 h. Each well was subsequently blocked by 1% casein in 1×PBS for 1 h, then added to 100 μL sGP7 phage supernatants. After 1 h, wells were washed by 10×0.05% PBST, added to 100 μL HRP-M13 major coat protein antibody (RL-ph1, Santa Cruz Biotechnology; 1:10,000 dilution with 1×PBS with 1% casein), and incubated at room temperature for 1 h. A colorimetric detection was performed using a 1-Step Ultra TMB ELISA substrate solution (Thermo Fisher Scientific) and OD450 was measured with a SpectraMax Plus 384 microplate reader (Molecular Devices). Limit of Detection (LoD) was calculated by mean blank+3×SD_{PS4}. More details on the SD selection is provided in (Tables 2 and 3).

Nanoparticle functionalization with nanobodies. The streptavidin surface functioned gold nanoparticles (typically ˜0.13 nM 80 nm AuNPs, 80 μL) were first mixed with an excessive amount of biotinylated nanobodies (about 1.2 μM, 25 μL). The mixture was then incubated for 2 hours to ensure complete streptavidin-biotin conjugation. Next, the mixture was purified by centrifuge (accuSpin Micro 17, Thermo Fisher) at 10,000 rpm for 10 min and repeated twice to remove unbounded biotinylated nanobodies. The purified AuNP colloid was measured by Nanodrop 2000 (Thermo Fisher) to determine the concentration. The concentration of AuNP in colloid was subsequently adjusted to get an optimal extinction level (e.g., empirically 0.048 nM for 80 nm AuNPs) and was aliquoted into 12 uL in a 500 uL Eppendorf tube.

Antigen detection. Target sGP or RBD stock solution (6 μM, in 1×PBS) underwent a 10-fold serial dilution to target concentrations (4 pM to 4 uM) in selected detection media. The final composition of PBS detection media was composed of 1×PBS, 20% v/v glycerol and 1 wt % BSA while that of FBS (Fetal Bovine Serum), HPS (Human Pooled Serum), and WB (Whole blood) detection media had a final concentration of 1×PBS, 20% v/v glycerol and 1 wt % BSA and 20% of either FBS, HPS, or WB, which resulted in a final concentration of FBS, HPS, or WB in the detection assay to be 5%. For example, for sGP detection with co-binders, solutions of sGP49-functionalized AuNPs, sGP7-functionalized AuNP, and sGP were mixed in a 500 uL Eppendorf tube at a ratio of 3:3:2 and thoroughly vortexed. When incubation-based detection was used, the solution was allowed to incubate (typically 3 hours) prior to readout.

Centrifuge enhanced rapid detection. The same protocols were followed in preparation of sGP49 surface functioned AuNP colloid. After mixing sGP49 surface functioned AuNP colloid with sGP solution, the AuNP assay colloid was centrifuged (accuSpin Micro 17, Thermo Fisher) at 3,500 rpm (1,200×g) for 1 minute. After optional incubation, the assay colloid was vortexed (mini vortexer, Thermo Fisher) at 800 rpm for 15 seconds prior to readout.

PDMS well plate fabrication. Sylgard 184 silicone elastomer base (consisting of dimethyl vinyl-terminated dimethyl siloxane, dimethyl vinylated, and trimethylated silica) was thoroughly mixed with the curing agent (mass ratio 10:1) for 30 minutes and placed in a vacuum container for 2 hours to remove the generated bubbles. The mixture was then poured into a flat plastic container at room temperature and incubated for one week, until the PDMS is fully cured. The PDMS membrane was then cut to rectangular shape, and 2 mm wells were drilled by punchers. To prevent non-specific bonding of proteins, the PDMS membrane was treated with PVA, adapted from methods described by Trantidou et al., Hydrophilic surface modification of PDMS for droplet microfluidics using a simple, quick, and robust method via PVA deposition. Microsystems & nanoengineering 2017, 3, 1-9. The as-prepared PDMS membrane and a diced rectangle shaped fused silica (500 μm thick) were both rinsed with isopropyl alcohol, dried in nitrogen, and treated by oxygen plasma (flow rate 2 sccm, power 75 W, 5 min). Immediately after, the two were bonded to form a PDMS well plate. The plate was further oxygen plasma treated for 5 min and immediately soaked in 1% wt. PVA in water solution for 10 min. Then, it was dried by nitrogen, heated on a 110° C. hotplate for 15 min, and cooled to room temperature by nitrogen blow.

UV-visible spectrometric measurement and dark field scattering characterizations. The UV-visible spectra and dark field imaging were performed using a customized optical system (Horiba), comprising an upright fluorescence microscope (Olympus BX53), a broadband 75 W Xenon lamp (PowerArc), an imaging spectrometer system (Horiba iHR320, spectral resolution 0.15 nm), a low-noise CCD spectrometer (Horiba Syncerity), a high-speed and low-noise EMCCD camera (Andor iXon DU897 Ultra), a vision camera, a variety of filter cubes, operation software, and a high-power computer. For spectral measurement, the PDMS plate loaded with upper-level assay samples or drop-cast samples on a glass slide was placed on the microscope sample stage. Light transmitted through PDMS well plate was collected by a 50× objective lens (NA=0.8). The focal plane was chosen at the well plate surface to display the best contrast at the hole edge. A 10× objective lens (NA=0.3) was used for drop-cast samples. The signals were typically collected from the 350 nm to 800 nm spectral range with integration time of 0.01 s and averaged 64 times. The dark field scattering was illuminated by an xeon lamp, collected by a 100× dark field lens (NA=0.9), and imaged by an EMCCD camera. The integration time was set to 50 ms. For each drop-cast sample spot, ten images were taken from different areas in the spot. The size for the area taken in each dark-field scattering image was 62.5 μm×62.5 μm.

SEM imaging of drop-cast samples on gold. The SEM image was taken by a Hitachi S4700 field emission scanning electron microscope at 5 kV acceleration voltage and magnification of 15,000. For each drop-cast sample spot, ten images were taken from different regions in the spot. The size of the region taken in each SEM image was 8.446 μm×5.913 μm.

TEM to image AuNP precipitates. The upper-level assay liquid was removed from the microcentrifuge tube until 2 to 3 μL of sample containing AuNP precipitates were left. The tube was vortexed thoroughly, and 2 μL of remaining samples were pipetted, and coated on a Cu grid (Electron Microscopy Sciences, C flat, hole size 1.2 μm, hole spacing 1.3 μm) that was pre-treated on both sides with oxygen plasma (30 seconds). The Cu grid was plunge frozen in ethane using Vitrobot plunge freezer (FEI). The blot time was set to 6 sec. After plunging, the sample was soaked in liquid nitrogen for long-term storage. FEI Tecnai F20 transmission electron microscope (200 kV accelerating voltage) was used for CryoTEM imaging. 25 high-resolution TEM images were taken for 1 μM, 1 nM sGP in PBS samples and reference samples, respectively. The size of the area taken in each image was 4.476 μm×4.476 μm.

Portable spectrometric readout. The assay colloids were initially characterized by a miniaturized portable UV-visible spectra measurement system. OSL2 fiber coupled illuminator (Thorlabs) was used as the light source. The light passed through the 4 mm diameter wells loaded with assay colloid and coupled to Flame UV-visible miniaturized spectrometer (Ocean optics) for extinction spectra measurement. The signals were averaged from six scans (each from 430 nm to 1100 nm) and integrated for 5 seconds in each scan.

Electronic readout with rapid test. A LED-photodiode electronic readout system consists of three key components: a LED light source, a photodiode, and a microcentrifuge tube holder. The centrifuge tube holder was 3D printed using ABSplus P430 thermoplastic. An 8.6 mm diameter recess was designed to snuggly fit a standard 0.5 mL Eppendorf tube. 2.8 mm diameter holes were open on two sides of the microcentrifuge tube holder to align a LED (597-3311-407NF, Dialight), the upper-level assay liquid, and a photodiode (SFH 2270R, Osram Opto Semiconductors). The LED was powered by two Duracell optimum AA batteries (3 V) through a serially connected 35Ω resistor to set the LED operating point. The photodiode was reversely biased by three Duracell optimum AA batteries (4.5 V) and serially connected to a 7 MΩ load resistor. The photocurrent that responds to intensity of light transmitted through the assay was converted to voltage through the 7 MΩ load resistor and measured with a portable multimeter (AstroAI AM33D).

Estimate of limit of detection for Nano2RED. In our work, limit of detection (LOD) was calculated according to the International Union of Pure and Applied Chemistry definition, that is, the concentration at which the measured response is able to distinguish from the reference signal by three times the standard deviation in measurements. For optical measurement, we used LoD=c(E_{NC}−3σ). Here, the reference was averaged over three measurements of the negative control (NC) sample. Whereas for electronic measurement, we used LoD=c(V_{NC}+3σ), where V_{NC} is the readout voltage for NC sample. We compared different methods to estimate a, i.e., the conventional way considering only the NC sample (σ_{NC}), using pooled sigma from all measurements (σ_{sA}), and pooled sigma from the four lowest concentrations (σ_{PS4}) (Table 2). We noticed in our case that using σ_{PSA} and σ_{PS4} both yielded more consistent reporting of LOD compared to using σ_{NC}, and therefore the sensor LOD was estimated with σ_{PSA} for its best consistency (Table 1). This consistency could be attributed to the nature of our data collection is a physical process and less dependent on reagent concentration compared to conventional ELISA. Particularly when using spectrometric readout, the noise is strongly affected by optical focusing and could happen to any data sets; and therefore the overall average provides a better estimate of the empirical errors. Differently, for ELISA measurement, the sigma is much smaller at lower concentrations, so we used conventional σ_{NC} for LOD determination.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, kits, reaction mixtures, devices, and/or systems or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A method of detecting Ebola virus in a sample, the method comprising:
contacting the sample with a plurality of gold nanoparticles (AuNPs) and/or other plasmonic metal nanoparticles (MNPs) that are conjugated with at least two sets of antibodies, or antigen binding portions thereof, wherein at least a first set of antibodies, or antigen binding portions thereof, binds to a first epitope of a glycoprotein from the Ebola virus and wherein at least a second set of antibodies, or antigen binding portions thereof, binds to a second epitope of a glycoprotein from the Ebola virus under conditions sufficient for the first and second set of antibodies, or the antigen binding portions thereof, to bind to the first or second epitopes of the glycoproteins from the Ebola virus in the sample to produce bound glycoproteins; or
contacting the sample with a plurality of gold nanoparticles (AuNPs) and/or other plasmonic metal nanoparticles (MNPs) that are conjugated with a single set of antibodies, or antigen binding portions thereof, that bind to an identical epitope from different monomers of a dimerized glycoprotein from the Ebola virus under conditions sufficient for the antibodies, or the antigen binding portions thereof, to bind to the identical epitope from the different monomers of the dimerized glycoprotein from the Ebola virus in the sample to produce bound glycoproteins; and,
detecting the glycoproteins from the Ebola virus when one or more aggregations of the bound glycoproteins form with one another, thereby detecting the Ebola virus in the sample.

2. The method of claim 1, wherein the antibodies comprise monoclonal antibodies.

3. The method of claim 1, wherein the first and second set of antibodies, or the antigen binding portions thereof, comprise nanobodies.

4. The method of claim 1, wherein the glycoprotein comprises a secreted glycoprotein (sGP).

5. The method of claim 1, wherein the detection step comprises determining a change in absorbance at a resonance wavelength of the AuNPs and/or the other MNPs.

6. The method of claim 1, comprising quantifying an amount of the glycoproteins and/or the Ebola virus in the sample.

7. The method of claim 1, further comprising centrifuging the aggregations of the bound glycoproteins prior to and/or during the detecting step.

8. The method of claim 1, further comprising freezing the aggregations of the bound glycoproteins prior to the detecting step.

9. The method of claim 1, comprising drop casting the aggregations of the bound glycoproteins prior to the detecting step.

10. The method of claim 1, comprising obtaining the sample from a subject.

11. The method of claim 10, comprising administering one or more therapies to the subject when the Ebola virus is detected in the sample.

12. The method of claim 10, comprising detecting the Ebola virus within about 20 minutes or less of obtaining the sample from the subject.

13. The method of claim 10, comprising repeating the method using one or more longitudinal samples obtained from the subject.

14. The method of claim 10, wherein the sample comprises blood, plasma, serum, saliva, sputum, or urine.

15. The method of claim 1, wherein the detecting step comprises measuring a colorimetric change when the one or more aggregations of the bound glycoproteins form with one another.

16. The method of claim 15, comprising visually detecting the colorimetric change when the one or more aggregations of the bound glycoproteins form with one another.

17. The method of claim 15, comprising detecting the colorimetric change when the one or more aggregations of the bound glycoproteins form with one another using a spectrometer.

18. A device, comprising at least one reaction chamber or substrate comprising a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a glycoprotein from an Ebola virus and wherein at least a second set of antibodies, or antigen binding portions thereof, binds to a second epitope of a glycoprotein from the Ebola virus when the reaction chamber or substrate receives a sample that comprises the Ebola virus under conditions sufficient for the first and second set of antibodies, or the antigen binding portions thereof, to bind to the first or second epitopes of the glycoproteins from the Ebola virus in the sample to produce bound glycoproteins and one or more aggregations of the bound glycoproteins to produce a colorimetric change in the reaction chamber; or (ii) conjugated with a single set of antibodies, or antigen binding portions thereof, that bind to an identical epitope from different monomers of a dimerized glycoprotein from the Ebola virus under conditions sufficient for the antibodies, or the antigen binding portions thereof, to bind to the identical epitope from the different monomers of the dimerized glycoprotein from the Ebola virus in the sample to produce bound glycoproteins and one or more aggregations of the bound glycoproteins to produce a colorimetric change in the reaction chamber.

19. A system, comprising:
a device, comprising at least one reaction chamber or substrate comprising a plurality of gold nanoparticles (AuNPs) and/or other plasmonic metal nanoparticles (MNPs) that are (i) conjugated with at least two sets of antibodies, or antigen binding portions thereof, wherein at least a first set of antibodies, or antigen binding portions thereof, binds to a first epitope of a glycoprotein from an Ebola virus and wherein at least a second set of antibodies, or antigen binding portions thereof, binds to a second epitope of a glycoprotein from the Ebola virus when the reaction chamber receives a sample that comprises the Ebola virus under conditions sufficient for the first and second set of antibodies, or the antigen binding portions thereof, to bind to the first or second epitopes of the glycoproteins from the Ebola virus in the sample to produce bound glycoproteins; or (ii) conjugated with a single set of antibodies, or antigen binding portions thereof, that bind to an identical epitope from different monomers of a dimerized glycoprotein from the Ebola virus when the reaction chamber receives a sample that comprises the Ebola virus under conditions sufficient for the antibodies, or the antigen binding portions thereof, to bind to the identical epitope from the different monomers of the dimerized glycoprotein from the Ebola virus in the sample to produce bound glycoproteins; and,
an electromagnetic radiation detection apparatus positioned, or positionable, within sufficient proximity to the device to detect one or more colorimetric changes produced in or on the reaction chamber or substrate when one or more aggregations of the bound glycoproteins form with one another in or on the reaction chamber or substrate.

20. The system of claim 19, wherein the electromagnetic radiation detection apparatus comprises a spectrometer; wherein the electromagnetic radiation detection apparatus comprises a microscope; and/or wherein the electromagnetic radiation detection apparatus comprises a light-emitting diode (LED) that transmits light into and/or through the reaction chamber or substrate and a photodetector that detects light from the reaction chamber or substrate.
